(19) United States
(12) Patent Application Publication
(10) Pub. No.: US 2004/0034021 A1
(43) Pub. Date: Feb. 19, 2004

(54) Intravenous rifalazil formulation and methods of use thereof

(76) Inventors: Arthur F. Michaelis, Devon, PA (US); Chalom Sayada, Luxembourg City (LU); Bernard E. Cabana, Montgomery Village, MD (US)

Correspondence Address: CLARK & ELBING LLP, 101 FEDERAL STREET, BOSTON, MA, 02110, US

(21) Appl. No.: 10/453,155

(22) Filed: Jun. 3, 2003

Related U.S. Application Data
(60) Provisional application No. 60/385,532, filed on Jun. 3, 2002.
(60) Provisional application No. 60/406,873, filed on Aug. 29, 2002.
(60) Provisional application No. 60/412,958, filed on Sep. 23, 2002.
(60) Provisional application No. 60/444,570, filed on Feb. 3, 2003.

(51) Int. Cl. A61K 31/538

(52) U.S. Cl. 514/229.5

(57) ABSTRACT

The invention features intravenous dosage formulations of rifalazil and methods of treating disease by intravenous administration of rifalazil.

### CROSS-REFERENCE TO RELATED APPLICATIONS

The application claims benefit of U.S. Provisional Application Nos. 60/385,532, filed Jun. 3, 2002, 60/406,873, filed Aug. 29, 2002, 60/412,958, filed Sep. 23, 2002, and 60/444,570, filed Feb. 3, 2003, each of which is herein incorporated by reference.

### BACKGROUND OF THE INVENTION

The invention relates to the fields of antimicrobial agents, formulations, and methods of treating disease.

In the last decade, the frequency and spectrum of antimicrobial-resistant infections has increased. Certain infections that are essentially untreatable are reaching epidemic proportions in both the developing world and institutional settings in the developed world. Antimicrobial resistance is manifested in increased morbidity, mortality, and health-care costs. Staphylococcus aureus is a significant cause of nosocomial and community acquired infections, especially skin and soft tissue infection, including surgical wound infection, nosocomial pneumonia, and bloodstream infection (see, for example, Panlilio et al., Infect. Cont. Hosp. Epidemiol. 13: 582-586 (1992)). Other pathogens commonly associated with serious infections include, but are not limited to, Staphylococcus spp., Streptococcus spp., Enterococcus spp., and Enterobacter spp. There exists a need to provide alternative and improved agents for the treatment of bacterial infections particularly for the treatment of infections caused by resistant strains of bacteria such as penicillin-resistant, methicillin-resistant (e.g., methicillin-resistant Staphylococcus aureus), quinolone-resistant (e.g., quinolone-resistant Streptococcus pneumoniae), macrolide-resistant (e.g., macrolide-resistant Streptococcus pyogenes), and/or vancomycin-resistant (e.g., vancomycin-resistant enterococci) strains (see, for example, Swartz M. N., N. Engl. J. Med. 346:722 (2002); Davidson et al., N. Engl. J. Med. 346:747 (2002); and Huovinen P., N. Engl. J. Med. 346:1243 (2002)). A considerable amount of effort has been devoted to developing antibacterial (bacteriostatic and/or bactericidal) agents with activity against these and other microorganisms.

One agent capable of treating a wide variety of infections is rifalazil. Rifalazil is described in the U.S. Pat. No. 4,983,602, where its antibacterial activity is disclosed.

### SUMMARY OF THE INVENTION

We have discovered methods of formulating rifalazil for intravenous administration, as well as developing compositions thereof, and methods of treating disease by administering rifalazil intravenously.

In one aspect, the invention features an aqueous solution of rifalazil suitable for intravenous administration to a human, wherein the solution has a rifalazil concentration of between 10 and 10,000 μg/mL. Desirably, the solution has a rifalazil concentration of between 10 and 5,000, 10 and 3,000, 50 and 10,000, 50 and 2,000, 100 and 10,000, 100 and 2,000, or 10 and 500 μg/mL.

The aqueous solution of rifalazil may contain one or more excipients. Particular excipients that may be used in the preparation of rifalazil solutions include polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, and ionic surfactants. Any excipient described herein can be used in the formulation of rifalazil. Desirably, the aqueous solutions of rifalazil include one or more excipients selected from sodium lauryl sulfate, polyoxyl-40 stearate, PEG-3 castor oil, PEG-5 castor oil, PEG-9 castor oil, PEG-16 castor oil, PEG-20 castor oil, PEG-23 castor oil, PEG-30 castor oil, PEG-35 castor oil, PEG-38 castor oil, PEG-40 castor oil, PEG-50 castor oil, PEG-60 castor oil, PEG-100 castor oil, PEG-200 castor oil, PEG-5 hydrogenated castor oil, PEG-7 hydrogenated castor oil, PEG-10hydrogenated castor oil, PEG-20 hydrogenated castor oil, PEG-25 hydrogenated castor oil, PEG-30 hydrogenated castor oil, PEG-40 hydrogenated castor oil, PEG-45 hydrogenated castor oil, PEG-50 hydrogenated castor oil, PEG-60 hydrogenated castor oil, PEG-80 hydrogenated castor oil, and PEG-100 hydrogenated castor oil.

The invention also features an aqueous composition for inhibiting the hydrolytic degradation of rifalazil dissolved therein. The composition includes rifalazil, water, and a micelle-forming excipient.

In addition, the invention features a method of treating disease in a human. This method includes the intravenous administration of an aqueous solution of rifalazil to a human in an amount effective to treat the disease. The aqueous solution of rifalazil is formulated as described herein and is suitable for administration to a human.

The methods of the invention can be used to treat any disease or infection for which rifalazil is effective including, for example, community-acquired pneumonia, upper and lower respiratory tract infection, skin and soft tissue infection, bone and joint infection, hospital-acquired lung infection, acute bacterial otitis media, bacterial pneumonia, complicated infection, noncomplicated infection, pyelonephritis, intra-abdominal infection, deep-seated abcess, bacterial sepsis, central nervous system infection, bacteremia, wound infection, peritonitis, meningitis, infections after burn, urogenital tract infection, gastro-intestinal tract infection, pelvic inflammatory disease, endocarditis, and intravascular infection.

The methods of the invention can also be used to treat diseases associated with bacterial infection. For example, bacterial infections can produce inflammation resulting in the pathogenesis of atherosclerosis, multiple sclerosis, rheumatoid arthritis, diabetes, Alzheimer's disease, asthma, cirrhosis of the liver, psoriasis, meningitis, cystic fibrosis, cancer, and osteoporosis. Accordingly, the invention features a method of treating such diseases, among others, by administering rifalazil intravenously.

The invention also includes the preoperative intravenous administration of rifalazil to reduce or eliminate the incidence of postoperative infections in patients undergoing surgical procedures or implantation of prosthetic devices.

In another aspect, the invention features a method of treating a non-mycobacterial infection by Gram-positive bacteria in a human patient by administering rifalazil to the patient in an amount effective to treat the infection. The Gram-positive bacterial infections to be treated include, without limitation, infections by, Staphylococcus aureus, Staphylococcus epidermidis, Enterococcus faecalis, Enterococcus faecium, Clostridium perfringens, Clostridium difficile, Streptococcus pyogenes, Streptococcus pneumoniae, other Streptococcus ssp., and other Clostridium spp.

In another aspect, the invention features a method of treating an infection by multi-drug resistant bacteria in a human by intravenous administration of rifalazil to the human in an amount effective to treat the infection. Resistant strains of bacteria include penicillin-resistant, methicillin-resistant, quinolone-resistant, macrolide-resistant, and/or vancomycin-resistant bacterial strains. The multi-drug resistant bacterial infections to be treated using the methods of the invention include, for example, infections by penicillin-, methicillin-, macrolide-, vancomycin-, and/or quinolone-resistant Streptococcus pneumoniae; penicillin-, methicillin-, macrolide-, vancomycin-, and/or quinolone-resistant Staphylococcus aureus; penicillin-, methicillin-, macrolide-, vancomycin-, and/or quinolone-resistant Streptococcus pyogenes; and penicillin-, methicillin-, macrolide-, vancomycin-, and/or quinolone-resistant enterococci.

The methods of the invention can further be used to treat or prevent infections by bacteria from a variety of genera, such as Escherichia spp., Enterobacter spp., Enterobacteriaceae spp., Klebsiella spp., Serratia spp., Pseudomonas spp., Acinetobacter spp., Bacillus spp., Micrococcus spp., Arthrobacter spp., Peptostreptococcus spp., Staphylococcus spp., Enterococcus spp., Streptococcus spp., Haemophilus spp., Neisseria spp., Bacteroides spp., Citrobacter spp., Branhamella spp., Salmonella spp., Shigella spp., Proteus spp., Clostridium spp., Erysipelothrix spp., Listeria spp., Pasteurella spp., Streptobacillus spp., Spirinurm spp., Fusospirocheta spp., Treponema spp., Borrelia spp., Actinomycetes spp., Mycoplasma spp., Chlamydia spp., Rickettsia spp., Spirochaeta spp., Legionella spp., Mycobacteria spp., Ureaplasma spp., Streptomyces spp., and Trichomoras spp. In this method, intravenous rifalazil is administered to the patient in an amount effective to treat or ameliorate the bacterial infection, or is administered prophylactically to prevent or reduce infection.

The invention further features a method of treating an intracellular infection by a facultative or obligate intracellular microbe. The method includes the intravenous administration of rifalazil in an amount effective to treat the intracellular infection. The microbe can be a bacterium, fungus, protozoan, or virus. Infections by the intracellular organisms described herein can be treated using this method.

In addition, the invention features a method for treating a human patient diagnosed as being infected with a bacterium having a multiplying form and a non-multiplying form by administering to the patient (i) rifalazil intravenously, and (ii) a second antibiotic that is effective against the multiplying form of the bacterium, wherein the two antibiotics are administered in amounts and for a duration that, in combination, treat the patient.

In one preferred method of carrying out the foregoing method, the antibiotic that is effective against the multiplying form of the bacterium is administered in an amount and for a duration to reduce the number of bacteria in the patient to less than about 10⁶ organisms/mL. This typically takes from a few hours to 1, 2, or 3 days, but may take as long as a week. After this has been achieved, the patient is then administered rifalazil intravenously in an amount and for a duration effective to complete the treatment of the patient. Antibiotics that are effective against the multiplying form of the bacterium include any of the antibiotics described herein.

The invention further features a method of treating a human patient diagnosed as having a chronic disease associated with a bacterial infection caused by bacteria capable of establishing a cryptic phase. The method includes the step of administering rifalazil intravenously to the patient.

The invention yet further features a method of treating the cryptic phase of a bacterial infection. This method includes the step of administering intravenous rifalazil to a patient. The administering is for a time and in an amount effective to treat the cryptic phase of the bacterial infection.

The invention also features a method of treating a bacterial infection in a human patient by (a) treating the multiplying form of the bacteria by administering an antibiotic to the patient for a time and an amount effective to treat the multiplying form, and (b) treating the non-multiplying form of the bacteria by administering rifalazil intravenously to the patient, wherein the administering is for a time and in an amount effective to treat the non-multiplying form.

Preferably, the bacterial infection is caused by one of the following: Chlamydia spp. (e.g., C. trachomatis, C. pneumoniae, C. psittaci, C. suis, C. ecorum, C. abortus, C. caviae, C. felis, C. muridarum), N. hartmannellae, W hondrophila, S. negevensis, or P. acanthamoeba.

The time effective to treat a cryptic phase or other non-multiplying form of a bacterium ranges from one day to one year. In certain instances, treatment can be for several weeks or months, or even extended over the lifetime of the individual patient, if necessary. For example, the duration of treatment may be at least 30 days, at least 45 days, at least 90 days, or at least 180 days. Ultimately, it is most desirable to extend the treatment for such a time that the non-multiplying form is no longer detectable.

The invention also features a method for treating or preventing the development of an atherosclerosis-associated disease in a human patient. The method includes the intravenous administration of rifalazil in an amount effective to treat or prevent the development of the atherosclerosis-associated disease in the patient. The patient is typically diagnosed as having the atherosclerosis-associated disease (or being at increased risk of developing the disease) or as having macrophages or foam cells infected with C. pneumoniae prior to the intravenous administration of rifalazil.

The invention also features a method of reducing the level of C-reactive protein in a human patient in need thereof. This method includes the intravenous administration of rifalazil in an amount effective to reduce the level of C-reactive protein in the patient. In one embodiment, the patient has not been diagnosed as having a bacterial infection. In another embodiment, the patient has been diagnosed as having macrophages or foam cells infected with C. pneumoniae.

The invention also features a method for reducing C. pneumoniae replication in macrophages or foam cells in a human patient in need thereof. This method includes the intravenous administration of rifalazil in an amount effective to reduce C. pneumoniae replication in macrophages or foam cells in the patient.

The invention also features a method for treating a persistent C. pneumoniae infection in macrophages or foam cells in a human patient. The method includes the intravenous administration of rifalazil in an amount effective to treat the C. pneumoniae infection in macrophages or foam cells in the patient.

The invention also features a method for treating a chronic disease associated with an infection of C. pneumoniae. This method includes intravenous administration of rifalazil in an amount effective to treat the infection.

The invention also features a method for treating a human patient having antibiotic-associated bacterial diarrhea or an infection of C. difficile, or preventing the disease or infection in the patient. The method includes the intravenous administration of rifalazil to the patient in an amount effective to treat the infection. The method may be employed as an initial treatment of a patient having or being at risk for developing antibiotic-associated bacterial diarrhea or infection of C. difficile, or it may be employed to treat patients for whom the initial treatment (e.g., with metronidazole or vancomycin) has failed to fully treat the antibiotic-associated bacterial diarrhea or an infection of C. difficile. The method may be employed, for example, when the patient is colonized with C. difficile organisms that are resistant to one or more of metronidazole, vancomycin, and rifampicin.

In any of the above treatment or prevention methods, rifalazil is administered intravenously. The intravenously administered rifalazil is formulated as an aqueous that includes rifalazil at a concentration of between 10 and 10,000 μg/mL, water, and one or more solubility enhancing pharmaceutically acceptable excipients.

If desired, rifalazil may be administered in conjunction with one or more additional agents such as anti-inflammatory agents (e.g., non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate) and steroids (e.g., cortisone, dexamethasone, hydrocortisone, methylprednisolone, prednisolone, prednisone, triamcinolone)), antibacterial agents (e.g., aminoglycosides, amphenicols, ansamycins, β-Lactams, carbapenems, cephalosporins, cephamycins, lincosamides, macrolides, polypeptides, tetracyclines, 2,4-diaminopyrimidines, nitrofurans, quinolones, sulfonamides, lipopeptides, oxazolidones, ketolides, or sulfones. Exemplary antibiotics include amikacin, gentamicin, kanamycin, tetracycline, vancomycin, teicoplanin, azithromycin, clarithromycin, erythromycin, gatifloxacin, levofloxacin, amoxicillin, and metronidazole), platelet aggregation inhibitors (e.g., abciximab, aspirin, cilostazol, clopidogrel, dipyridamole, eptifibatide, ticlopidine, or tirofiban), anticoagulants (e.g., dalteparin, danaparoid, enoxaparin, heparin, tinzaparin, or warfarin), antipyretics (e.g., acetaminophen), or lipid lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These additional agents may be administered within 14 days, 7 days, 1 day, 12 hours, or 1 hour of the intravenous administration of rifalazil, or simultaneously therewith. The additional therapeutic agents may be present in the same or different pharmaceutical compositions as the intravenous formulation of rifalazil. When present in different pharmaceutical compositions, different routes of administration may be used. For example, a second agent may be administered orally or by intramuscular or subcutaneous injection. Agents that can be administered in conjunction with rifalazil include any of the agents described herein.

For any of the methods described herein, rifalazil can be administered by intravenous infusion, wherein between 1 and 48 mg of rifalazil is administered over a period of 4 to 24 hours. Desirably, between 1 and 40 mg, 1 and 30 mg, 2 and 30 mg, 3 and 30 mg, or 4 and 25 mg of rifalazil is administered over a period of 4 to 24 hours, 8 to 24 hours, or 15 to 24 hours. Up to 2, 4, 6, 8, 10, 12, 14, 16, 18, 20, 22, 24, 26, 28, 30, 32, 34, 36, 38, 40, 42, 44, 46, 48, or 50 mg of rifalazil is administered by intravenous infusion over a 2, 4, 5, 6, 7, 8, 9, 10, 12, 14, 20, 24, 48, or 72 hour period.

For any of the methods described herein, rifalazil can be administered by intravenous bolus of between 2 and 25 mg of rifalazil over a 10 to 60 minute period followed by a slow infusion of 0.1 to 2 mg, 0.5 to 2 mg, 0.5 to 1.5 mg, or 1 to 2 mg, per hour for up to 24 hours.

The intravenous administration of rifalazil may be repeated as needed. For example, the administration may be repeated daily, or every other day, for a period of two to fourteen days, or can be repeated every third day for a period of three to fifteen days, or once weekly for a period of three to sixteen weeks.

In another aspect, the invention features a method of treating disease in a human. The method includes the intravenous administration of rifalazil at a rate that maintains a plasma concentration of rifalazil of between 2 and 100, 2 and 80, 2 and 60, 2 and 30, 6 and 50, or 10 and 50 ng/mL for a period greater than 5, 8, 12, or 24 hours.

Desirably, rifalazil is administered in a dosing regimen that maintains a plasma concentration of rifalazil of between 2 and 100, 2 and 60, or 2 and 40 ng/mL for a period greater than 24 hours.

The invention also features a pharmaceutical formulation for intravenous administration including rifalazil. The formulation includes an aqueous solution of rifalazil and is packaged with a label or package insert providing instructions for the use of the formulation wherein the instructions describe an intravenous dosing regimen.

The compositions can also be packaged as a concentrate including rifalazil and micelle-forming excipient. The concentrate optionally includes some water. For example, the concentrate can be less than 40%, 20%, 10%, 5%, or even 1% water by volume. The concentrate contains greater than 100 μg/mL, 1 mg/mL, 5 mg/mL, 10 mg/mL, or 20 mg/mL of rifalazil.

As used herein, the term “treating” refers to administering a pharmaceutical composition for prophylactic and/or therapeutic purposes. To “prevent disease” refers to prophylactic treatment of a human patient who is not yet ill, but who is susceptible to, or otherwise at risk of, a particular disease. To “treat disease” or use for “therapeutic treatment” refers to administering treatment to a patient already suffering from a disease to improve or stabilize the patient's condition. Thus, in the claims and embodiments, treating is the administration to a human patient either for therapeutic or prophylactic purposes.

As used herein, the term “administration” or “administering” refers to a method of giving a dosage of a pharmaceutical composition to a human.

By “effective” amount is meant the amount of rifalazil required to treat or prevent an infection or a disease associated with an infection. The effective amount of rifalazil used to practice the invention for therapeutic or prophylactic treatment of conditions caused by or contributed to by a microbial infection varies depending upon the manner of administration, the age, body weight, and general health of the subject. Ultimately, the attending physician will decide the appropriate amount and dosage regimen. Such amount is referred to as an “effective” amount.

By “aqueous solution” is meant a water-based liquid that is greater than 40% water by volume and without undissolved solids above 0.5 microns in size. Desirably, the aqueous solutions of rifalazil include greater than 60%, 70%, 80%, 90%, 95%, 97%, or even 98% water (w/w) and the rifalazil is completely dissolved. Rifalazil can be dissolved in either an aqueous phase or a micellar phase of the aqueous solution.

By “micellar phase” is meant the hydrophobic interior of an aggregate micelle) containing lipophilic (e.g., surfactant) molecules.

As used in herein, a “micelle-forming excipient” refers to an excipient dissolved in an aqueous solution in an amount sufficient to form micelles within the solution at 25° C. The formation of micelles can be monitored using any of several standard techniques known in the art, including surface tension measurements, solubilization of water insoluble dye, conductivity measurements, and light scattering, among others.

By “an aqueous composition for inhibiting the hydrolytic decomposition of rifalazil” is meant an aqueous solution in which less than ten percent of the rifalazil is degraded to des-acetyl rifalazil at 25° C. over a one year period.

As used herein, “suitable for intravenous administration to a human” refers to an aqueous solution including rifalazil and one or more pharmaceutically acceptable excipients. Solutions that are suitable for intravenous administration to a human do not include excipients that would compromise the health of a patient. For example, certain organic solvents (e.g., dimethyl sulfoxide, ethanol, propanol, acetone, and dimethyl formamide) are miscible in water and useful for the preparation of aqueous solutions of insoluble compounds. However, these organic solvents are poisons in the amounts required for the formulation of rifalazil and, therefore, could not be administered intravenously to a patient without compromising the health of the patient. Furthermore, solutions that are suitable for intravenous administration to a human have a pH of between 4 and 9.

By “bolus” injection or administration is meant intravenous administration of rifalazil wherein a dose of greater than 2 mg of rifalazil is administered over a period of less than one hour.

By “infusion” is meant a continuous intravenous administration of rifalazil over a period of greater than one hour wherein rifalazil is administered at a constant rate of less than or equal to 2 mg of rifalazil per hour.

By “atherosclerosis” is meant the progressive accumulation of smooth muscle cells, immune cells (e.g., lymphocytes, macrophages, or monocytes), lipid products (e.g., lipoproteins, or cholesterol), cellular waste products, calcium, or other substances within the inner lining of an artery, resulting in the narrowing or obstruction of the blood vessel and the development of atherosclerosis-associated diseases. Atherosclerosis is typically manifested within large and medium-sized arteries, and is often characterized by a state of chronic inflammation within the arteries.

By “atherosclerosis-associated disease” is meant any disorder that is caused by or is associated with atherosclerosis. Typically, atherosclerosis of the coronary arteries commonly causes coronary artery disease, myocardial infarction, coronary thrombosis, and angina pectoris. Atherosclerosis of the arteries supplying the central nervous system frequently provokes strokes and transient cerebral ischemia. In the peripheral circulation, atherosclerosis causes intermittent claudication and gangrene and can jeopardize limb viability. Atherosclerosis of an artery of the splanchnic circulation can cause mesenteric ischemia. Atherosclerosis can also affect the kidneys directly (e.g., renal artery stenosis).

A human patient who is being treated for an atherosclerosis-associated disease is one who a medical practitioner has diagnosed as having such a disease. Diagnosis may be by any suitable means. Methods for diagnosing atherosclerosis by measuring systemic inflammatory markers are described, for example, in U.S. Pat. No. 6,040,147, hereby incorporated by reference. Diagnosis and monitoring may employ an electrocardiogram, chest X-ray, echocardiogram, cardiac catheterization, ultrasound (for the measurement of vessel wall thickness), or measurement of blood levels of CPK, CPK-MB, myoglobin, troponin, homocysteine, or C-reactive protein. A patient in whom the development of an atherosclerosis-associated disease is being prevented is one who has not received such a diagnosis. One in the art will understand that these patients may have been subjected to the same tests (electrocardiogram, chest X-ray, etc.) or may have been identified, without examination, as one at high risk due to the presence of one or more risk factors (e.g., family history, hypertension, diabetes mellitus, high cholesterol levels). Thus, prophylactic intravenous administration of rifalazil is considered to be preventing the development of an atherosclerosis-associated disease.

An atherosclerosis-associated disease has been treated or prevented when one or more tests of the disease (e.g., any of the those described above) indicate that the patient's condition has improved or the patient's risk reduced. In one example, a reduction in C-reactive protein to normal levels indicates that an atherosclerosis-associated disease has been treated or prevented.

An alternative means by which treatment or prevention is assessed includes determination of the presence of an infection of C. pneumoniae. Any suitable method may be employed (e.g., determination of C. pneumoniae in blood monocytes or in the atheroma itself (e.g., in macrophages or foam cells present in the fatty streak), or detection of C. pneumoniae DNA, RNA, or antibodies to C. pneumoniae in a biological sample from the patient).

“Antibiotic-associated bacterial diarrhea” means the condition wherein antibiotic therapy disturbs the balance of the microbial flora of the gut, allowing pathogenic organisms such as C. difficile to flourish. These organisms cause diarrhea. Antibiotic-associated bacterial diarrhea includes such conditions as C. difficile associated diarrhea (CDAD) and pseudomembranous colitis. When rifalazil is administered intravenously for the treatment of a C. difficile infection, an effective amount of rifalazil is the amount required to eradicate C. difficile from the patient, or the amount which prevents an infection of C. difficile, as determined by a diagnostic test that detects C. difficile.

“Pseudomembranous colitis,” also known as pseudomembranous enterocolitis or enteritis, means the inflammation of the mucous membrane of both small and large intestine with the formation and passage of pseudomembranous material (composed of fibrin, mucous, necrotic epithelial cells and leukocytes) in the stools.

The term “lower gastrointestinal tract” means the lower part of the small intestine (ileum) and the colon.

By “autoimmune disease” is meant a disease arising from an immune reaction against self-antigens and directed against the individual's own tissues. Examples of autoimmune diseases include but are not limited to systemic lupus erythematosus, rheumatoid arthritis, myasthenia gravis, and Graves' disease.

By “bacteria” is meant a unicellular prokaryotic microorganism that usually multiplies by cell division.

By “bacteria capable of establishing a cryptic phase” is meant any species whose life cycle includes a persistent, non-multiplying phase. These species include but are not limited to C. trachomatis, C. pneumoniae, C. psittaci, C. suis, C. pecorum, C. abortus, C. caviae, C. felis, C. muridarum, N. hartmannellae, W. chondrophila, S. negevensis, and P. acanthamoeba, as well as any other species described in Everett et al. (Int. J. Syst. Evol. Microbiol. 49:415-440, (1999)).

By “bacterial infection” is meant the invasion of a host by pathogenic bacteria. For example, the infection may include the excessive growth of bacteria that are normally present in or on the body of a human or growth of bacteria that are not normally present in or on a human. More generally, a bacterial infection can be any situation in which the presence of a bacterial population(s) is damaging to a host body. Thus, a human is “suffering” from a bacterial infection when an excessive amount of a bacterial population is present in or on the person's body, or when the presence of a bacterial population(s) is damaging the cells or other tissue of the person.

By “chronic disease” is meant an inveterate disease of long continuance, or which progresses slowly, in contrast to an acute disease, which rapidly terminates. A chronic disease may begin with a rapid onset or in a slow insidious manner but it tends to persist for several weeks, months or years, and has a vague and indefinite termination.

By “cryptic phase” is meant the latent or dormant intracellular phase of infection characterized by little or no metabolic activity. The non-replicating cryptic phase is often characteristic of persistent forms of intracellular bacterial infections.

By “elementary body phase” is meant the infectious phase of the bacterial life cycle which is characterized by the presence of elementary bodies (EBs). EBs are small (300-400 nm), infectious, spore-like forms which are metabolically inactive, non-replicating, and found most often in the acellular milieu. EBs possess a rigid outer membrane which protects them from a variety of physical insults such as enzymatic degradation, sonication and osmotic pressure.

By “immunocompromised” is meant a person who exhibits an attenuated or reduced ability to mount a normal cellular or humoral defense to challenge by infectious agents, e.g., viruses, bacterial, fungi, and protozoa. Persons considered immunocompromised include malnourished patients, patients undergoing surgery and bone narrow transplants, patients undergoing chemotherapy or radiotherapy, neutropenic patients, HIV-infected patients, trauma patients, burn patients, patients with chronic or resistant infections such as those resulting from myelodysplastic syndrome, and the elderly, all of who may have weakened immune systems.

By “inflammatory disease” is meant a disease state characterized by (1) alterations in vascular caliber that lead to an increase in blood flow, (2) structural changes in the microvasculature that permit the plasma proteins and leukocytes to leave the circulation, and (3) emigration of the leukocytes from the microcirculation and their accumulation in the focus of injury. The classic signs of acute inflammation are erythema, edema, tenderness (hyperalgesia), and pain. Chronic inflammatory diseases are characterized by infiltration with mononuclear cells (e.g., macrophages, lymphocytes, and plasma cells), tissue destruction, and fibrosis. Non-limiting examples of inflammatory disease include asthma, coronary artery disease, arthritis, conjunctivitis, lymphogranuloma venerum, and salpingitis.

By “intracytoplasmic inclusion” is meant a replicating reticulate body (RB) that has no cell wall. Such inclusions may be detected, for example, through chlamydiae sample isolation and propagation on a mammalian cell lines, followed by fixing and staining using one of a variety of staining methods including Giemsa staining, iodine staining, and immunofluorescence. These inclusions have a typical round or oval appearance.

By “persistent bacterial infection” is meant an infection that is not completely eradicated through standard treatment regimens using antibiotics. Persistent bacterial infections are caused by bacteria capable of establishing a cryptic phase or other non-multiplying form of a bacterium and may be classified as such by culturing bacteria from a patient and demonstrating bacterial survival in vitro in the presence of antibiotics or by determination of anti-bacterial treatment failure in a patient. As used herein, a persistent infection in a patient includes any recurrence of an infection, after receiving antibiotic treatment, from the same species more than two times over the period of two or more years or the detection of the cryptic phase of the infection in the patient. An in vivo persistent infection can be identified through the use of a reverse transcriptase polymerase chain reaction (RT-PCR) to demonstrate the presence of 16S rRNA transcripts in bacterially infected cells after treatment with one or more antibiotics (Antimicrob. Agents Chemother. 12:3288-3297, (2000)).

By “replicating phase” is meant the phase of the bacterial cell cycle characterized by the presence of an RB. The RB is the actively replicating form of the Chlamydia. It contains no cell wall and is detected as an inclusion in the cell.

The term “microbial infection” refers to the invasion of the host animal by pathogenic microbes. This includes the excessive growth of microbes that are normally present in or on the body of an animal. More generally, a microbial infection can be any situation in which the presence of a microbial population(s) is damaging to a host animal. Thus, an animal is “suffering” from a microbial infection when excessive numbers of a microbial population are present in or on an animal's body, or when the presence of a microbial population(s) is damaging the cells or other tissue of an animal.

The term “microbes” includes, for example, bacteria, fungi, yeasts, viruses and protozoa.

By “intracellular pathogen” is meant an infection by any facultative or obligate intracellular microbe.

By “obligate intracellular pathogen” is meant a microbe which must use an intracellular location (e.g., a host cell) in order to replicate.

By “facultative intracellular pathogen” is meant a microbe which is able to survive within an intracellular location (e.g., a host cell), but does not require an intracellular environment to replicate.

### BRIEF DESCRIPTION OF THE DRAWINGS

FIG. 1 is a graph of the solubility of rifalazil in water as a function of pH.

FIG. 2 is a graph depicting the solubility of rifalazil in solvent-water mixtures.

FIG. 3 is a graph depicting the influence of solubilizing agents on the solubility of rifalazil in water.

FIG. 4 is a graph depicting the solubility of rifalazil in aqueous solutions containing lipophilic salts. DTAB=dodecyltrimethylammonium bromide; Cheno=sodium chenodeoxycholate; Octyl=sodium octylsulfate; Deoxy=sodium deoxycholate; Cholate=sodium cholate; SDS=sodium dodecylsulfate.

FIG. 5 is a graph depicting the solubility of rifalazil in aqueous solutions containing varying amounts of sodium dodecylsulfate at pH 5.4 and 7.4.

FIG. 6 is a graph depicting the solubility of rifalazil in aqueous solutions containing varying amounts of PEG-35 castor oil.

FIG. 7 is a graph depicting the hydrolytic degradation of rifalazil in the presence of the micelle-forming excipient PEG-35 castor oil as a function of time.

FIG. 8 is a graph depicting the hydrolytic degradation of rifalazil in the absence of a micelle-forming excipient as a function of time.

### DETAILED DESCRIPTION

In general, the invention provides aqueous solutions of rifalazil that are suitable for intravenous administration to a human. The aqueous solutions include one or more excipients that enhance the solubility and inhibit the hydrolytic degradation of rifalazil.

For the treatment of many nosocomial and serious community acquired infections, it is often desirable to administer rifalazil parenterally, because of the lack of predictability in the bioavailability of orally administered rifalazil to diseased individuals. Intravenous administration is preferred for the treatment of life-threatening infections, for patients with severe illness, for persistent infections, and for prophylaxis against postoperative infections in patients undergoing surgical procedures.

Formulation

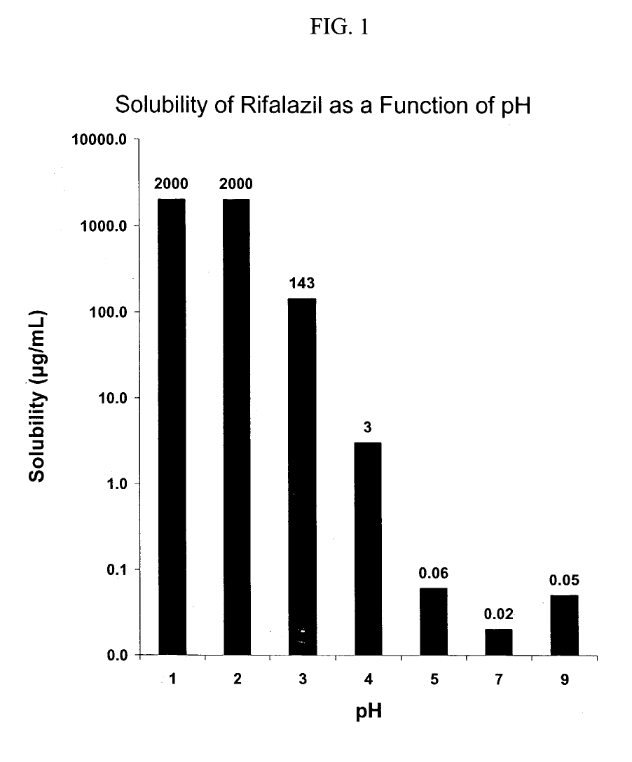

Rifalazil is virtually insoluble in water at physiological pH. A typical low dosage concentration of rifalazil for intravenous administration is 100 μg/mL, which is 5,000 times greater than the solubility of the drug in water at pH 7 (see FIG. 1). In order to provide a reasonable safety margin for an intravenous dosage form of rifalazil, the target solubility at room temperature, allowing for solubility changes due to extremes of temperature, is set at a value five to ten times higher, or 0.5 to 1.0 mg/mL.

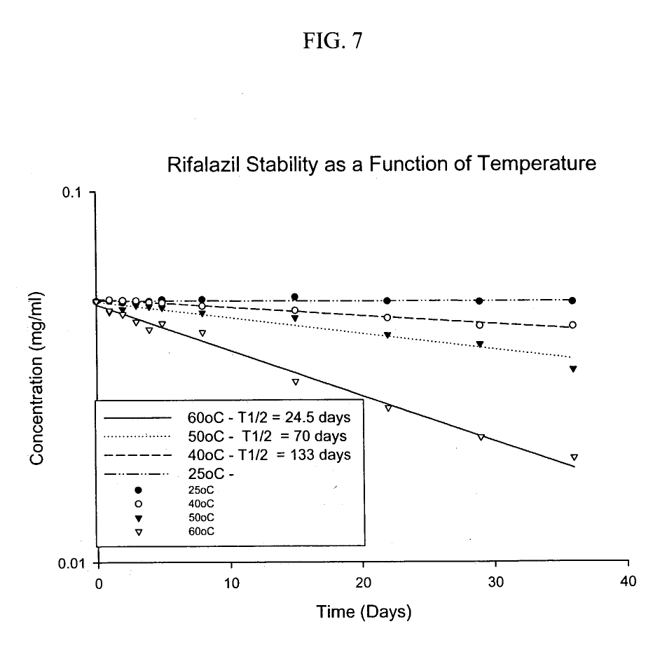

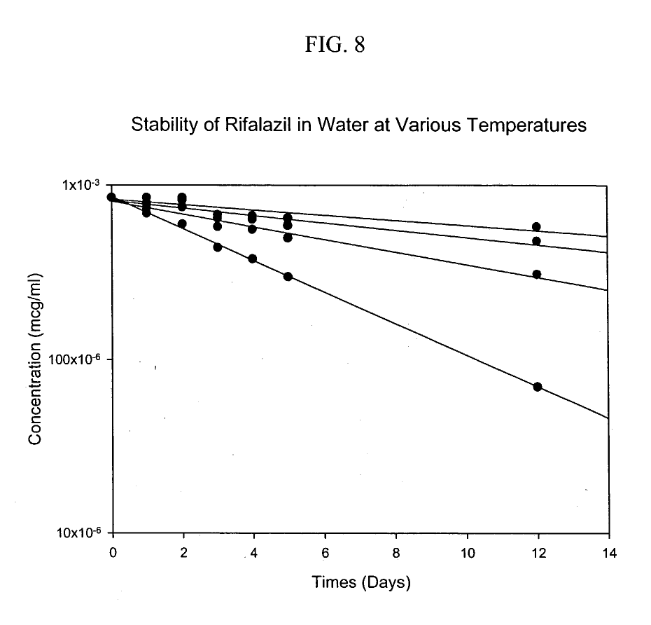

Another challenge to the use of aqueous formulations of rifalazil is the hydrolytic degradation of rifalazil, which occurs readily in aqueous environments under ambient conditions (see FIG. 8 and Example 3). To be commercially useful, any pharmaceutical formulation must maintain the active ingredient in a stable and predictable form prior to administration to a human. The formulations described herein overcome the hydrolytic degradation of rifalazil by the addition of a micelle-forming excipient, which inhibits the degradation of rifalazil (see FIG. 7 and Example 3) in comparison to aqueous solutions in the absence of a micelle-forming excipient.

Solubilizing excipients can be used for the preparation of an intravenous dosage formulation of rifalazil. The excipients used are restricted to those that have a high degree of safety in humans.

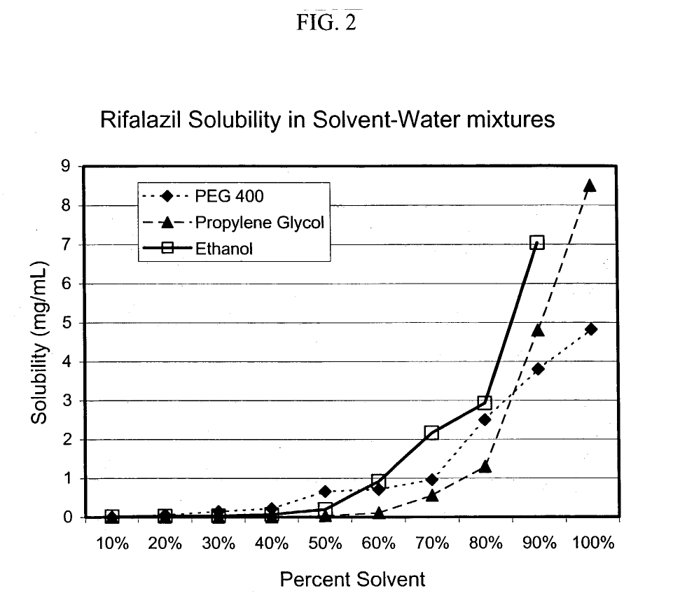

As used herein, “solubilization” describes the improvement in the solubility of rifalazil resulting from the addition of surface-active compounds to the aqueous solution. The solubilizates formed contain rifalazil present in dissolved form in the molecular associations, micelles, of the surface-active compounds, which form in aqueous solution (see FIGS. 2-5). The resulting solutions appear optically clear to opalescent.

A variety of solubilizers may be used for the formulation of rifalazil including those solubilizers disclosed in U.S. Pat. No. 6,365,637, herein incorporated by reference, proteins which readily bind lipophilic compounds such as albumin, and compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, and ionic surfactants. Commercially available examples for each class of excipient are provided below.

Polyethoxylated fatty acids may be used as excipients for the formulation of rifalazil. Examples of commercially available polyethoxylated fatty acid monoester surfactants include: PEG 4-100 monolaurate (Crodet L series, Croda), PEG 4-100 monooleate (Crodet 0 series, Croda), PEG 4-100 monostearate (Crodet S series, Croda, and Myrj Series, Atlas/ICI), PEG 400 distearate (Cithrol 4DS series, Croda), PEG 100, 200, or 300 monolaurate (Cithrol ML series, Croda), PEG 100, 200, or 300 monooleate (Cithrol MO series, Croda), PEG 400 dioleate (Cithrol 4DO series, Croda), PEG 400-1000 monostearate (Cithrol MS series, Croda), PEG-1 stearate (Nikkol MYS-lEX, Nikko, and Coster Kl, Condea), PEG-2 stearate (Nikkol MYS-2, Nikko), PEG-2 oleate (Nikkol MYO-2, Nikko), PEG-4 laurate (Mapeg® 200 ML, PPG), PEG-4 oleate (Mapeg® 200 MO, PPG), PEG-4 stearate (Kessco® PEG 200 MS, Stepan), PEG-5 stearate (Nikkol TMGS-5, Nikko), PEG-5 oleate (Nikkol TMGO-5, Nikko), PEG-6 oleate (Algon OL 60, Auschem SpA), PEG-7 oleate (Algon OL 70, Auschem SpA), PEG-6 laurate (Kessco® PEG300 ML, Stepan), PEG-7 laurate (Lauridac 7, Condea), PEG-6 stearate (Kessco® PEG300 MS, Stepan), PEG-8 laurate (Mapeg® 400 ML, PPG), PEG-8 oleate (Mapeg® 400 MO, PPG), PEG-8 stearate (Mapeg® 400 MS, PPG), PEG-9 oleate (Emulgante A9, Condea), PEG-9 stearate (Cremophor S9, BASF), PEG-10 laurate (Nikkol MYL-10, Nikko), PEG-10 oleate (Nikkol MYO-10, Nikko), PEG-12 stearate (Nikkol MYS-10, Nikko), PEG-12 laurate (Kessco® PEG 600 ML, Stepan), PEG-12 oleate (Kessco® PEG 600 MO, Stepan), PEG-12 ricinoleate (CAS #9004-97-1), PEG-12 stearate (Mapeg® 600 MS, PPG), PEG-15 stearate (Nikko® TMGS-15, Nikko), PEG-15 oleate (Nikkol TMGO-15, Nikko), PEG-20 laurate (Kessco® PEG 1000 ML, Stepan), PEG-20 oleate (Kessco® PEG 1000 MO, Stepan), PEG-20 stearate (Mapeg® 1000 MS, PPG), PEG-25 stearate (Nikkol MYS-25, Nikko), PEG-32 laurate (Kessco® PEG 1540 ML, Stepan), PEG-32 oleate (Kessco® PEG 1540 MO, Stepan), PEG-32 stearate (Kessco® PEG 1540 MS, Stepan), PEG-30 stearate (Myrj 51), PEG-40 laurate (Crodet L40, Croda), PEG-40 oleate (Crodet 040, Croda), PEG-40 stearate (Emerest® 2715, Henkel), PEG-45 stearate (Nikkol MYS-45, Nikko), PEG-50 stearate (Myrj 53), PEG-55 stearate (Nikkol MYS-55, Nikko), PEG-100 oleate (Crodet O-100, Croda), PEG-100 stearate (Ariacel 165, ICI), PEG-200 oleate (Albunol 200 MO, Taiwan Surf.), PEG-400 oleate (LACTOMUL, Henkel), and PEG-600 oleate (Albunol 600 MO, Taiwan Surf.). Formulations of rifalazil according to the invention may include one or more of the polyethoxylated fatty acids above.

Polyethylene glycol fatty acid diesters may also be used as excipients for the formulation of rifalazil. Examples of commercially available polyethylene glycol fatty acid diesters include: PEG-4 dilaurate (Mapeg® 200 DL, PPG), PEG-4 dioleate (Mapeg® 200 DO, PPG), PEG-4 distearate (Kessco® 200 DS, Stepan), PEG-6 dilaurate (Kessco® PEG 300 DL, Stepan), PEG-6 dioleate (Kessco® PEG 300 DO, Stepan), PEG-6 distearate (Kessco® PEG 300 DS, Stepan), PEG-8 dilaurate (Mapeg® 400 DL, PPG), PEG-8 dioleate (Mapeg® 400 DO, PPG), PEG-8 distearate (Mapeg® 400 DS, PPG), PEG-10 dipalmitate (Polyaldo 2PKFG), PEG-12 dilaurate (Kessco® PEG 600 DL, Stepan), PEG-12 distearate (Kessco® PEG 600 DS, Stepan), PEG-12 dioleate (Mapeg® 600 DO, PPG), PEG-20 dilaurate (Kessco® PEG 1000 DL, Stepan), PEG-20 dioleate (Kessco® PEG 1000 DO, Stepan), PEG-20 distearate (Kessco® PEG 1000 DS, Stepan), PEG-32 dilaurate (Kessco® PEG 1540 DL, Stepan), PEG-32 dioleate (Kessco® PEG 1540 DO, Stepan), PEG-32 distearate (Kessco® PEG 1540 DS, Stepan), PEG-400 dioleate (Cithrol 4DO series, Croda), and PEG-400 distearate Cithrol 4DS series, Croda). Formulations of rifalazil according to the invention may include one or more of the polyethylene glycol fatty acid diesters above.

PEG-fatty acid mono- and di-ester mixtures may be used as excipients for the formulation of rifalazil. Examples of commercially available PEG-fatty acid mono- and di-ester mixtures include: PEG 4-150 mono, dilaurate (Kessco® PEG 200-6000 mono, Dilaurate, Stepan), PEG 4-150 mono, dioleate (Kessco® PEG 200-6000 mono, Dioleate, Stepan), and PEG 4-150 mono, distearate (Kessco® 200-6000 mono, Distearate, Stepan). Formulations of rifalazil according to the invention may include one or more of the PEG-fatty acid mono- and di-ester mixtures above.

In addition, polyethylene glycol glycerol fatty acid esters may be used as excipients for the formulation of rifalazil. Examples of commercially available polyethylene glycol glycerol fatty acid esters include: PEG-20 glyceryl laurate (Tagat® L, Goldschmidt), PEG-30 glyceryl laurate (Tagat® L2, Goldschmidt), PEG-15 glyceryl laurate (Glycerox L series, Croda), PEG-40 glyceryl laurate (Glycerox L series, Croda), PEG-20 glyceryl stearate (Capmul® EMG, ABITEC), and Aldo® MS-20 KFG, Lonza), PEG-20 glyceryl oleate (Tagat® O, Goldschmidt), and PEG-30 glyceryl oleate (Tagat® O2, Goldschmidt). Formulations of rifalazil according to the invention may include one or more of the polyethylene glycol glycerol fatty acid esters above.

Alcohol-oil transesterification products may also be used as excipients for the formulation of rifalazil. Examples of commercially available alcohol-oil transesterification products include: PEG-3 castor oil (Nikkol CO-3, Nikko), PEG-5, 9, and 16 castor oil (ACCONON CA series, ABITEC), PEG-20 castor oil, (Emalex C-20, Nihon Emulsion), PEG-23 castor oil (Emulgante EL23), PEG-30 castor oil (Incrocas 30, Croda), PEG-35 castor oil (Incrocas-35, Croda), PEG-38 castor oil (Emulgante EL 65, Condea), PEG-40 castor oil (Emalex C-40, Nihon Emulsion), PEG-50 castor oil (Emalex C-50, Nihon Emulsion), PEG-56 castor oil (Eumulgin® PRT 56, Pulcra SA), PEG-60 castor oil (Nikkol CO-60TX, Nikko), PEG-100 castor oil, PEG-200 castor oil (Eumulgin® PRT 200, Pulcra SA), PEG-5 hydrogenated castor oil (Nikkol HCO-5, Nikko), PEG-7 hydrogenated castor oil (Cremophor WO7, BASF), PEG-10 hydrogenated castor oil (Nikkol HCO-10, Nikko), PEG-20 hydrogenated castor oil (Nikkol HCO-20, Nikko), PEG-25 hydrogenated castor oil (Simulsol® 1292, Seppic), PEG-30 hydrogenated castor oil (Nikkol HCO-30, Nikko), PEG-40 hydrogenated castor oil (Cremophor RH 40, BASF), PEG-45 hydrogenated castor oil (Cerex ELS 450, Auschem Spa), PEG-50 hydrogenated castor oil (Emalex HC-50, Nihon Emulsion), PEG-60 hydrogenated castor oil (Nikkol HCO-60, Nikko), PEG-80 hydrogenated castor oil (Nikkol HCO-80, Nikko), PEG-100 hydrogenated castor oil (Nikkol HCO-100, Nikko), PEG-6 corn oil (Labrafil® M 2125 CS, Gattefosse), PEG-6 almond oil (Labrafil® M 1966 CS, Gattefosse), PEG-6 apricot kernel oil (Labrafil® M 1944 CS, Gattefosse), PEG-6 olive oil (Labrafil® M 1980 CS, Gattefosse), PEG-6 peanut oil (Labrafil® M 1969 CS, Gattefosse), PEG-6 hydrogenated palm kernel oil (Labrafil® M 2130 BS, Gattefosse), PEG-6 palm kernel oil (Labrafil® M 2130 CS, Gattefosse), PEG-6 triolein (Labrafil® M 2735 CS, Gattefosse), PEG-8 corn oil (Labrafil® WL 2609 BS, Gattefosse), PEG-20 corn glycerides (Crovol M40, Croda), PEG-20 almond glycerides (Crovol A40, Croda), PEG-25 trioleate (TAGAT® TO, Goldschmidt), PEG-40 palm kernel oil (Crovol PK-70), PEG-60 corn glycerides (Crovol M70, Croda), PEG-60 almond glycerides (Crovol A70, Croda), PEG-4 caprylic/capric triglyceride (Labrafac® Hydro, Gattefosse), PEG-8 caprylic/capric glycerides (Labrasol, Gattefosse), PEG-6 caprylic/capric glycerides (SOFTIGEN®767, Huls), lauroyl macrogol-32 glyceride (GELUCIRE 44/14, Gattefosse), stearoyl macrogol glyceride (GELUCIRE 50/13, Gattefosse), mono, di, tri, tetra esters of vegetable oils and sorbitol (SorbitoGlyceride, Gattefosse), pentaerythrityl tetraisostearate (Crodamol PTIS, Croda), pentaerythrityl distearate (Albunol DS, Taiwan Surf.), pentaerythrityl tetraoleate (Liponate PO-4, Lipo Chem.), pentaerythrityl tetrastearate (Liponate PS-4, Lipo Chem.), pentaerythrityl tetracaprylate tetracaprate (Liponate PE-810, Lipo Chem.), and pentaerythrityl tetraoctanoate (Nikkol Pentarate 408, Nikko). Also included as oils in this category of surfactants are oil-soluble vitamins, such as vitamins A, D, E, K, etc. 15 Thus, derivatives of these vitamins, such as tocopheryl PEG 1000 succinate (TPGS, available from Eastman), are also suitable surfactants. Formulations of rifalazil according to the invention may include one or more of the alcohol-oil transesterification products above.

Polyglycerized fatty acids may also be used as excipients for the formulation of rifalazil. Examples of commercially available polyglycerized fatty acids include: polyglyceryl-2 stearate (Nikkol DGMS, Nikko), polyglyceryl-2 oleate (Nikkol DGMO, Nikko), polyglyceryl-2 isostearate (Nikkol DGMIS, Nikko), polyglyceryl-3 oleate (Caprol® 3GO, ABITEC), polyglyceryl-4 oleate (Nikkol Tetraglyn 1-O, Nikko), polyglyceryl-4 stearate (Nikkol Tetraglyn 1-S, Nikko), polyglyceryl-6 oleate (Drewpol 6-1-O, Stepan), polyglyceryl-10 laurate (Nikkol Decaglyn 1-L, Nikko), polyglyceryl-10 oleate (Nikkol Decaglyn 1-O, Nikko), polyglyceryl-10 stearate (Nikkol Decaglyn 1-S, Nikko), polyglyceryl-6 ricinoleate (Nikkol Hexaglyn PR-15, Nikko), polyglyceryl-10 linoleate (Nikkol Decaglyn 1-LN, Nikko), polyglyceryl-6 pentaoleate (Nikkol Hexaglyn 5-O, Nikko), polyglyceryl-3 dioleate (Cremophor GO32, BASF), polyglyceryl-3 distearate (Cremophor GS32, BASF), polyglyceryl-4 pentaoleate (Nikkol Tetraglyn 5-O, Nikko), polyglyceryl-6 dioleate (Caprol® 6G20, ABITEC), polyglyceryl-2 dioleate (Nikkol DGDO, Nikko), polyglyceryl-10 trioleate (Nikkol Decaglyn 3-O, Nikko), polyglyceryl-10 pentaoleate (Nikkol Decaglyn 5-O, Nikko), polyglyceryl-10 septaoleate (Nikkol Decaglyn 7-O, Nikko), polyglyceryl-10 tetraoleate (Caprol® 10G40, ABITEC), polyglyceryl-10 decaisostearate (Nikkol Decaglyn 10-IS, Nikko), polyglyceryl-101 decaoleate (Drewpol 10-10-O, Stepan), polyglyceryl-10 mono, dioleate (Caprol® PGE 860, ABITEC), and polyglyceryl polyricinoleate (Polymuls, Henkel). Formulations of rifalazil according to the invention may include one or more of the polyglycerized fatty acids above.

In addition, propylene glycol fatty acid esters may be used as excipients for the formulation of rifalazil. Examples of commercially available propylene glycol fatty acid esters include: propylene glycol monocaprylate (Capryol 90, Gattefosse), propylene glycol monolaurate (Lauroglycol 90, Gattefosse), propylene glycol oleate (Lutrol OP2000, BASF), propylene glycol myristate (Mirpyl), propylene glycol monostearate (LIPO PGMS, Lipo Chem.), propylene glycol hydroxystearate, propylene glycol ricinoleate (PROPYMULS, Henkel), propylene glycol isostearate, propylene glycol monooleate (Myverol P-06, Eastman), propylene glycol dicaprylate dicaprate (Captex® 200, ABITEC), propylene glycol dioctanoate (Captex® 800, ABITEC), propylene glycol caprylate caprate (LABRAFAC PG, Gattefosse), propylene glycol dilaurate, propylene glycol distearate (Kessco® PGDS, Stepan), propylene glycol dicaprylate (Nikkol Sefsol 228, Nikko), and propylene glycol dicaprate (Nikkol PDD, Nikko). Formulations of rifalazil according to the invention may include one or more of the propylene glycol fatty acid esters above.

Mixtures of propylene glycol esters and glycerol esters may also be used as excipients for the formulation of rifalazil. One preferred mixture is composed of the oleic acid esters of propylene glycol and glycerol (Arlacel 186). Examples of these surfactants include: oleic (ATMOS 300, ARLACEL 186, ICI), and stearic (ATMOS 150). Formulations of rifalazil according to the invention may include one or more of the mixtures of propylene glycol esters and glycerol esters above.

Further, mono- and diglycerides may be used as excipients for the formulation of rifalazil. Examples of commercially available mono- and diglycerides include: monopalmitolein (C16:1) (Larodan), monoelaidin (C18:1) (Larodan), monocaproin (C6) (Larodan), monocaprylin (Larodan), monocaprin (Larodan), monolaurin (Larodan), glyceryl monomyristate (C14) (Nikkol MGM, Nikko), glyceryl monooleate (C18:1) (PECEOL, Gattefosse), glyceryl monooleate (Myverol, Eastman), glycerol monooleate/linoleate (OLICINE, Gattefosse), glycerol monolinoleate (Maisine, Gattefosse), glyceryl ricinoleate (Softigen® 701, Huls), glyceryl monolaurate (ALDO® MLD, Lonza), glycerol monopalmitate (Emalex GMS-P, Nihon), glycerol monostearate (Capmul® GMS, ABITEC), glyceryl mono- and dioleate (Capmul® GMO-K, ABITEC), glyceryl palmitic/stearic (CUTINA MD-A, ESTAGEL-G18), glyceryl acetate (Lameging EE, Grunau GmbH), glyceryl laurate (Imwitor® 312, Huls), glyceryl citrate/lactate/oleate/linoleate (Imwitor® 375, Huls), glyceryl caprylate (Imwitor® 308, Huls), glyceryl caprylate/caprate (Capmul® MCM, ABITEC), caprylic acid mono- and diglycerides (Imwitorg® 988, Huls), caprylic/capric glycerides (Imwitor® 742, Huls), Mono-and diacetylated monoglycerides (Myvacet® 9-45, Eastman), glyceryl monostearate (Aldo® MS, Arlacel 129, ICI), lactic acid esters of mono and diglycerides (LAMEGIN GLP, Henkel), dicaproin (C6) (Larodan), dicaprin (C10) (Larodan), dioctanoin (C8) (Larodan), dimyristin (C14) (Larodan), dipalmitin (C16) (Larodan), distearin (Larodan), glyceryl dilaurate (C 12) (Capmul® GDL, ABITEC), glyceryl dioleate (Capmul® GDO, ABITEC), glycerol esters of fatty acids (GELUCIRE 39/01, Gattefosse), dipalmitolein (C16:1) (Larodan), 1,2 and 1,3-diolein (C18:1) (Larodan), dielaidin (C18:1) (Larodan), and dilinolein (C 18:2) (Larodan). Formulations of rifalazil according to the invention may include one or more of the mono- and diglycerides above.

Sterol and sterol derivatives may also be used as excipients for the formulation of rifalazil. Examples of commercially available sterol and sterol derivatives include: cholesterol, sitosterol, lanosterol, PEG-24 cholesterol ether (Solulan C-24, Amerchol), PEG-30 cholestanol (Phytosterol GENEROL series, Henkel), PEG-25 phytosterol (Nikkol BPSH-25, Nikko), PEG-5 soyasterol (Nikkol BPS-5, Nikko), PEG-10 soyasterol (Nikkol BPS-10, Nikko), PEG-20 soyasterol (Nikkol BPS-20, Nikko), and PEG-30 soyasterol (Nikkol BPS-30, Nikko). Formulations of rifalazil according to the invention may include one or more of the sterol and sterol derivatives above.

Polyethylene glycol sorbitan fatty acid esters may also be used as excipients for the formulation of rifalazil. Examples of commercially available polyethylene glycol sorbitan fatty acid esters include: PEG-10 sorbitan laurate (Liposorb L-10, Lipo Chem.), PEG-20 sorbitan monolaurate (Tween® 20, Atlas/ICI), PEG-4 sorbitan monolaurate (Tween® 21, Atlas/ICI), PEG-80 sorbitan monolaurate (Hodag PSML-80, Calgene), PEG-6 sorbitan monolaurate (Nikkol GL-1, Nikko), PEG-20 sorbitan monopalmitate (Tween® 40, Atlas/ICI), PEG-20 sorbitan monostearate (Tween® 60, Atlas/ICI), PEG-4 sorbitan monostearate (Tween® 61, Atlas/ICI), PEG-8 sorbitan monostearate (DACOL MSS, Condea), PEG-6 sorbitan monostearate (Nikkol TS 106, Nikko), PEG-20 sorbitan tristearate (Tween® 65, Atlas/ICI), PEG-6 sorbitan tetrastearate (Nikkol GS-6, Nikko), PEG-60 sorbitan tetrastearate (Nikkol GS-460, Nikko), PEG-5 sorbitan monooleate (Tween® 81, Atlas/ICI), PEG-6 sorbitan monooleate (Nikkol TO-106, Nikko), PEG-20 sorbitan monooleate (Tween® 80, Atlas/ICI), PEG-40 sorbitan oleate (Emalex ET 8040, Nihon Emulsion), PEG-20 sorbitan trioleate (Tween® 85, Atlas/ICI), PEG-6 sorbitan tetraoleate (Nikkol GO-4, Nikko), PEG-30 sorbitan tetraoleate (Nikkol GO-430, Nikko), PEG-40 sorbitan tetraoleate (Nikkol GO-440, Nikko), PEG-20 sorbitan monoisostearate (Tween® 120, Atlas/ICI), PEG sorbitol hexaoleate (Atlas G-1086, ICI), polysorbate 80 (Tween® 80, Pharma), polysorbate 85 (Tween® 85, Pharma), polysorbate 20 (Tween® 20, Pharma), polysorbate 40 (Tween® 40, Pharma), polysorbate 60 (Tween® 60, Pharma), and PEG-6 sorbitol hexastearate (Nikkol GS-6, Nikko). Formulations of rifalazil according to the invention may include one or more of the polyethylene glycol sorbitan fatty acid esters above.

In addition, polyethylene glycol alkyl ethers may be used as excipients for the formulation of rifalazil. Examples of commercially available polyethylene glycol alkyl ethers include: PEG-2 oleyl ether, oleth-2 (Brij 92/93, Atlas/ICI), PEG-3 oleyl ether, oleth-3 (Volpo 3, Croda), PEG-5 oleyl ether, oleth-5 (Volpo 5, Croda), PEG-10 oleyl ether, oleth-10 (Volpo 10, Croda), PEG-20 oleyl ether, oleth-20 (Volpo 20, Croda), PEG-4 lauryl ether, laureth-4 (Brij 30, Atlas/ICI), PEG-9 lauryl ether, PEG-23 lauryl ether, laureth-23 (Brij 35, Atlas/ICI), PEG-2 cetyl ether (Brij 52, ICI), PEG-10 cetyl ether (Brij 56, ICI), PEG-20 cetyl ether (BriJ 58, ICI), PEG-2 stearyl ether (Brij 72, ICI), PEG-10 stearyl ether (Brij 76, ICI), PEG-20 stearyl ether (Brij 78, ICI), and PEG-100 stearyl ether (Brij 700, ICI). Formulations of rifalazil according to the invention may include one or more of the polyethylene glycol alkyl ethers above.

Sugar esters may also be used as excipients for the formulation of rifalazil. Examples of commercially available sugar esters include: sucrose distearate (SUCRO ESTER 7, Gattefosse), sucrose distearate/monostearate (SUCRO ESTER 11, Gattefosse), sucrose dipalmitate, sucrose monostearate (Crodesta F-160, Croda), sucrose monopalmitate (SUCRO ESTER 15, Gattefosse), and sucrose monolaurate (Saccharose monolaurate 1695, Mitsubisbi-Kasei). Formulations of rifalazil according to the invention may include one or more of the sugar esters above. polyethylene glycol alkyl phenols are also useful as excipients for the formulation of rifalazil. Examples of commercially available polyethylene glycol alkyl phenols include: PEG-10-100 nonylphenol series (Triton X series, Rohm & Haas) and PEG-15-100 octylphenol ether series (Triton N-series, Rohm & Haas). Formulations of rifalazil according to the invention may include one or more of the polyethylene glycol alkyl phenols above.

Polyoxyethylene-polyoxypropylene block copolymers may also be used as excipients for the formulation of rifalazil. These surfactants are available under various trade names, including one or more of Synperonic PE series (ICI), Pluronicg series (BASF), Lutrol (BASF), Supronic, Monolan, Pluracare, and Plurodac. The generic term for these copolymers is “poloxamer” (CAS 9003-11-6). These polymers have the formula I:

HO(C₂H₄O)ₐ(C₃H₆O)_{b}(C₂H₄O)ₐH I

where “a” and “b” denote the number of polyoxyethylene and polyoxypropylene units, respectively. These copolymers are available in molecular weights ranging from 1000 to 15000 daltons, and with ethylene oxide/propylene oxide ratios between 0.1 and 0.8 by weight. Formulations of rifalazil according to the invention may include one or more of the polyoxyethylene-polyoxypropylene block copolymers above.

Polyoxyethylenes, such as PEG 300, PEG 400, and PEG 600, may be used as excipients for the formulation of rifalazil.

Sorbitan fatty acid esters may also be used as excipients for the formulation of rifalazil. Examples of commercially sorbitan fatty acid esters include: sorbitan monolaurate (Span-20, Atlas/ICI), sorbitan monopalmitate (Span-40, Atlas/ICI), sorbitan monooleate (Span-80, Atlas/ICI), sorbitan monostearate (Span-60, Atlas/ICI), sorbitan trioleate (Span-85, Atlas/ICI), sorbitan sesquioleate (Arlacel-C, ICI), sorbitan tristearate (Span-65, Atlas/ICI), sorbitan monoisostearate (Crill 6, Croda), and sorbitan sesquistearate (Nikkol SS-15, Nikko). Formulations of rifalazil according to the invention may include one or more of the sorbitan fatty acid esters above.

Esters of lower alcohols (C₂ to C₄) and fatty acids (C₈ to C ₁₈) are suitable surfactants for use in the invention. Examples of these surfactants include: ethyl oleate (Crodamol EO, Croda), isopropyl myristate (Crodamol IPM, Croda), isopropyl palmitate (Crodamol IPP, Croda), ethyl linoleate (Nikkol VF-E, Nikko), and isopropyl linoleate (Nikkol VF-IP, Nikko). Formulations of rifalazil according to the invention may include one or more of the lower alcohol fatty acid esters above.

In addition, ionic surfactants may be used as excipients for the formulation of rifalazil. Examples of useful ionic surfactants include: sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate (dodecyl), sodium tetradecyl sulfate, sodium lauryl sarcosinate, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco cheno deoxycholate, sodium cholylsarcosinate, sodium N-methyl taurocholate, egg yolk phosphatides, hydrogenated soy lecithin, dimyristoyl lecithin, lecithin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, diethanolamine, phospholipids, polyoxyethylene-10 oleyl ether phosphate, esterification products of fatty alcohols or fatty alcohol ethoxylates, with phosphoric acid or anhydride, ether carboxylates (by oxidation of terminal OH group of, fatty alcohol ethoxylates), succinylated monoglycerides, sodium stearyl fumarate, stearoyl propylene glycol hydrogen succinate, mono/diacetylated tartaric acid esters of mono- and diglycerides, citric acid esters of mono-, diglycerides, glyceryl-lacto esters of fatty acids, acyl lactylates, lactylic esters of fatty acids, sodium stearoyl-2-lactylate, sodium stearoyl lactylate, alginate salts, propylene glycol alginate, ethoxylated alkyl sulfates, alkyl benzene sulfones, α-olefin sulfonates, acyl isethionates, acyl taurates, alkyl glyceryl ether sulfonates, sodium octyl sulfosuccinate, sodium undecylenamideo-MEA-sulfosuccinate, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine), lauryl betaine (N-lauryl,N,N-dimethylglycine), and ethoxylated amines (polyoxyethylene-15 coconut amine). For simplicity, typical counterions are provided above. It will be appreciated by one skilled in the art, however, that any bioacceptable counterion may be used. For example, although the fatty acids are shown as sodium salts, other cation counterions can also be used, such as, for example, alkali metal cations or ammonium. Formulations of rifalazil according to the invention may include one or more of the ionic surfactants above.

Many of the foregoing excipients are micelle-forming in aqueous solutions. Whether an excipient forms a micelle will depend upon a number of conditions, including the concentration of the excipient, the composition of the aqueous solution, and the temperature. The formation of micelles can be monitored using any of several standard techniques known in the art, including surface tension measurements, solubilization of water insoluble dye, conductivity measurements, and light scattering, among others. In all of these methods, an abrupt change in some physicochemical property is measured as a function of excipient concentration. The abrupt change occurs when the concentration of excipient is sufficient to form micelles. Above this concentration, also known as the critical micelle concentration (CMC), micelles are present in solution.

The excipients present in the formulations of the invention are present in amounts such that the carrier forms a clear, or opalescent, aqueous dispersion of rifalazil. The relative amount of an excipient necessary for the preparation of the solutions described herein is readily determined by observing the solubility of rifalazil in the solution. For example, the optical clarity of the aqueous dispersion can be measured using standard quantitative techniques for turbidity assessment.

Methods for making formulations for intravenous administration are found, for example, in “Remington: The Science and Practice of Pharmacy” (20th ed., ed. A. R. Gennaro AR., 2000, Lippincott Williams & Wilkins). Formulations for intravenous administration may, for example, contain any one or combination of the excipients described above, sterile water, isotonic saline, isotonic dextrose solution, or nutritional supplements such as glucose. Alternatively, nanoparticulate formulations (e.g., biodegradable nanoparticles, solid lipid nanoparticles) may be used to prepare an intravenous dosage form of rifalazil. Other potentially useful intravenous delivery systems include ethylene-vinyl acetate copolymer particles, osmotic pumps, implantable infusion systems, and liposomes.

Other Therapeutic Agents

Rifalazil formulations and compositions described herein may also include a second therapeutic agent, including for example, another antibiotic, an anesthetic, an antimicrobial agent, a zinc salt, or an anti-inflammatory agent (e.g., an non-steroidal anti-inflammatory or a steroid).

Antibiotics that can be admixed with the intravenous rifalazil formulation include: aminoglycosides, such as amikacin, apramycin, arbekacin, ambermycins, butirosin, dibekacin, dihydrostreptomycin, fortimicin(s), fradiomycin, gentamicin, ispamicin, kanamycin, micronomicin, neomycin, neomycin undecylenate, netilmicin, paromomycin, ribostamycin, sisomicin, spectinomycin, streptomycin, streptonicozid, and tobramycin; amphenicols, such as azidamfenicol, chloramphenicol, chloramphenicol palmirate, chloramphenicol pantothenate, florfenicol, and thiamphenicol; ansamycins, such as rifampin, rifabutin, rifapentine, and rifaximin; β-Lactams, such as amidinocillin, amdinocillin, pivoxil, amoxicillin, ampicillin, aspoxicillin, azidocillin, azlocillin, bacampicillin, benzylpenicillinic acid, benzylpenicillin, carbenicillin, carfecillin, carindacillin, clometocillin, cloxacillin, cyclacillin, dicloxacillin, diphenicillin, epicillin, fenbenicillin, floxicillin, hetacillin, lenampicillin, metampicillin, methicillin, mezlocillin, nafcillin, oxacillin, penamecillin, penethamate hydriodide, penicillin G benethamine, penicillin G benzathine, penicillin G benzhydrylamine, penicillin G calcium, penicillin G hydragamine, penicillin G potassium, penicillin G, procaine, penicillin N, penicillin O, penicillin V, penicillin V benzathine, penicillin V hydrabamine, penimepicycline, phenethicillin, piperacillin, pivapicillin, propicillin, quinacillin, sulbenicillin, talampicillin, temocillin and ticarcillin; carbapenems, such as imipenem; cephalosporins, such as 1-carba (dethia) cephalosporin, cefactor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpirimide, cefpodoxime proxetil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin, cephalothin, cefaclor, cefotetan, cefprozil, loracarbef, cefetamet, and cefepime; cephamycins such as cefbuperazone, cefmetazole, cefminox, cefetan, and cefoxitin; monobactams such as aztreonam, carumonam, and tigemonan; oxacephems such as flomoxef and moxolactam; lincosamides such as clindamycin and lincomycin; macrolides such as azithromycin, carbomycin, clarithromycin, crythromycin(s) and derivatives, josamycin, leucomycins, midecamycins, miokamycin, oleandomycin, primycin, rokitamycin, rosaramicin, roxithromycin, spiramycin and troleandomycin; polypeptides such as amphomycin, bacitracin, capreomycin, colistin, enduracidin, enylomycin, fusafungine, gramicidin(s), gramicidin S, mikamycin, polymyxin, polymyxin β-methanesulfonic acid, pristinamycin, ristocetin, teicoplanin, thiostrepton, tuberactinomycin, tyrocidine, tyrothricin, vancomycin, viomycin(s), virginiamycin and zinc bacitracin; tetracyclines such as spicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, senociclin and tetracycline; and 2,4-diaminopyrimidines such as brodimoprim, tetroxoprim and trimethoprim; nitrofurans such as furaltadone, furazolium, nifuradene, nifuratel, nifurfoline, nifurpirinol, nifurprazine, nifurtoinol and nitrofurantoin; quinolones such as amifloxacin, cinoxacin, ciprofloxacin, difloxacin, enoxacin, fleroxacin, flumequine, lomefloxacin, miloxacin, nalidixic acid, norfloxacin, ofloxacin, oxolinic acid, perfloxacin, pipemidic acid, piromidic acid, rosoxacin, temafloxacin, and tosufloxacin; sulfonamides such as acetyl sulfamethoxypyrazine, acetyl sulfisoxazole, azosulfamide, benzylsulfamide, chloramine-β, chloramine-T, dichloramine-T, formosulfathiazole, N₂-formyl-sulfisomidine, N₄-β-D-glucosylsulfanilamide, mafenide, 4′-(methyl-sulfamoyl) sulfanilanilide, p-nitrosulfathiazole, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, sulfanilamidomethanesulfonic acid triethanolamine salt, 4-sulfanilamidosalicyclic acid, N₄-sulfanilylsulfanilamide, sulfanilylurea, N-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine and sulfisoxazole; sulfones, such as acedapsone, acediasulfone, acetosulfone, dapsone, diathymosulfone, glucosulfone, solasulfone, succisulfone, sulfanilic acid, p-sulfanilylbenzylamine, p,p′-sulfonyldianiline-N, N′digalactoside, sulfoxone and thiazolsulfone; lipopeptides such as daptomycin; oxazolidones such as linezolid; ketolides such as telithromycin; and miscellaneous antibiotics such as clofoctol, hexedine, magainins, methenamine, methenamine anhydromethylene-citrate, methenamine hippurate, methenamine mandelate, methenamine sulfosalicylate, nitroxoline, squalamine, xibomol, cycloserine, mupirocin, and tuberin.

When admixing an antimicrobial agent, the antimicrobial agent is preferably amoxillin, erythromycin, azithromycin, clarithromycin, gentamicin, tobramycin, ciprofloxaxin, norfloxacin, gatifloxacin, ofloxacin, levofloxacin, moxifloxacin, metronidazole, lomefloxacin, ciprofloxacin, natamycin, neomycin, polymyxin B, gentamycin, bacitracin, trovafloxacin, grepafloxacin, sulfacetamide, tetracycline, gramicidin, chloremphenicol, or gramicidin.

Preferred non-steroidal anti-inflammatory agents include, for example, detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, and pseudoephedrine, and preferred steroids include, for example, hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone.

Preferred anesthetics include, for example, benzocaine, butamben picrate, tetracaine, dibucaine, prilocaine, etidocaine, mepivacaine, bupivicaine, and lidocaine.

Preferred zinc salts include, for example, zinc sulfate, zinc chloride, zinc acetate, zinc phenol sulfonate, zinc borate, zinc bromide, zinc nitrate, zinc glycerophosphate, zinc benzoate, zinc carbonate, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, and zinc glycinate.

All of the therapeutic agents employed in the compositions of the invention can be used in the dose ranges currently known and used for these agents. Different concentrations may be employed depending on the clinical condition of the patient, the goal of therapy (treatment or prophylaxis), the anticipated duration, and the severity of the infection or disease for which intravenous rifalazil is being administered. Additional considerations in dose selection include the type of infection, age of the patient (e.g., pediatric, adult, or geriatric), general health, and comorbidity. Determining what concentrations to employ are within the skills of the pharmacist, medicinal chemist, or medical practitioner formulating the intravenous rifalazil in combination with other therapeutic agents.

Administration

One of the advantages of the invention is that the intravenous dosage formulations provide clinicians with the ability to directly adjust the plasma levels of rifalazil to the point of therapeutic efficacy by controlling the dose and the schedule of drug administration. Adjusting the dose and schedule of drug administration as described herein can result in a superior ability to achieve a safer and more effective treatment of disease. Specifically, by avoiding high initial peak levels, peak blood level-related side effects are minimized or, in some instances, eliminated.

Rifalazil can be administered by intravenous infusion, wherein between 1 and 48 mg of rifalazil is administered over a period of 4 to 24 hours. Desirably, between 1 and 40 mg, 1 and 30 mg, 2 and 30 mg, 3 and 30 mg, or 4 and 25 mg of rifalazil is administered over a period of 4 to 24 hours, 8 to 24 hours, 15 to 24 hours, or 20 to 24 hours. Up to 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, or 50 mg of rifalazil is administered by intravenous infusion over a 2, 4, 5, 6, 7, 8, 9, 10, 12, 14, 20, 24, 48, or 72 hour period.

Alternatively, rifalazil can be administered by intravenous bolus followed by slow infustion. Desirably, a bolus injection of between 2 and 25 mg of rifalazil over a 10 to 60 minute period is followed by a slow infusion of 0.1 to 2 mg per hour for up to 24 hours.

The intravenous administration of rifalazil may be repeated daily or every other day, for a period of two to fourteen days. The intravenous administration may be repeated every third day for a period of three to fifteen days. Desirably, the intravenous administration is repeated once weekly for a period of three to sixteen weeks.

Adjusting the dose and schedule of drug administration as described herein, rifalazil can be intravenously administered at a rate that maintains a plasma concentration of rifalazil of between 2 and 100, 2 and 80, 2 and 60, 2 and 40, 2 and 30,2 and 20,2 and 15,2 and 10,4 and 50,4 and 30,4 and 20,6 and 50, 6 and 30, 12and30,6and25,8and20,9and20, 12and20,2and 10,4and 12, or 10 and 50 ng/mL for a period greater than 4, 5, 6, 7, 8, 9, 10, 11, 12, 13, 14, 15, 16, 17, 18, 19, 20, 21, 22, 23, 24, 32, 40, 48, or 72 hours.

Desirably, rifalazil is administered in a dosing regimen that maintains a plasma concentration of rifalazil of between 2 and 40 ng/mL for a period greater than 24 hours.

Therapy

The methods and compositions of the invention can be used to treat, for example, community-acquired pneumonia, upper and lower respiratory tract infection, skin and soft tissue infection, bone and joint infection, hospital-acquired lung infection, acute bacterial otitis media, bacterial pneumonia, complicated infection, noncomplicated infection, pyelonephritis, intra-abdominal infection, deep-seated abcess, bacterial sepsis, central nervous system infection, bacteremia, wound infection, peritonitis, meningitis, infections after burn, urogenital tract infection, gastro-intestinal tract infection, pelvic inflammatory disease, endocarditis, and intravascular infection.

Furthermore, the methods and compositions of the invention can be used to treat bacteria in their non-multiplying growth phase, non-multiplying bacteria, which typically survive standard antimicrobial therapy (see, e.g., Martinez et al., Antimicrob. Agents Chemother. 44:1771-1777 (2000); Riesenfeld et al., Antimicrob. Agents Chemother. 41:2059-2060 (1997); Alonso et al., Microbiology 145:2857-2862 (1999)).

The methods and compositions of the invention can be used to treat or prevent bacterial infections by facultative intracellular bacteria, such as Bordetella pertussis, B. parapertussis, B. bronchiseptica, Burkholderia cepacia, Escherichia coli, Haemophilus actinomycetemcomitans, H. aegyptius, H. aphrophilus, H. ducreyi, H. felis, H. haemoglobinophilus, H. haemolyticus, H. influenzae, H. paragallinarum, H. parahaemolyticus, H. parainfluenzae, H. paraphrohaemolyticus, H. paraphrophilus, H. parasuis, H. piscium, H. segnis, H. somnus, H. vaginalis, Legionella adelaidensis, L. anisa, L. beliardensis, L. birminghamensis, L. bozemanii, L. brunensis, L. cherrii, L. cincinnatiensis, Legionella drozanskii L. dumoffli, L. erythra, L. fairfieldensis, L. fallonii, L. eeleii, L. geestiana, L. gormanii, L. gratiana, L. gresilensis, L. hackeliae, L. israelensis, L. jordanis, L. lansingensis, Legionella londiniensis L. longbeachae, Legionella lytica L. maceachernii, L. micdadei, L. moravica, L. nautarum, L. oakridgensis, L. parisiensis, L. pittsburghensis, L. pneumophila, L. quateirensis, L. quinlivanii, L. rowbothamii, L. rubrilucens, L. sainthelensi, L. santicrucis, L. shakespearei, L. spiritensis, L. steigerwaltii, L. taurinensis, L. tucsonensis, L. wadsworthii, L. waltersii, L. worsleiensis, Listeria denitrificans, L. grayi, L. innocua, L. ivanovii, L. monocytogenes, L. seeligeri, L. welshimeri, Mycobacterium abscessus, M. africanum, M. agri, M. aichiense, M. alvei, M. asiaticum, M, aurum, M. austroafricanum, M. avium, M. bohemicum, M. bovis, M. branderi, M. brumae, M. celatum, M. chelonae, M. chitae, M. chlorophenolicum, M. chubuense, M. confluentis, M. conspicuum, M. cookii, M. diernhoferi, M. doricum, M. duvalii, M. elephantis, M. fallax, M. farcinogenes, M. flavescens, M. ortuitum, M. frederiksbergense, M. gadium, M. gastri, M. genavense, M. gilvum, M. goodii, M. gordonae, M. haemophilum, M. hassiacum, M. heckeshornense, M. heidelbergense, M. hiberniae, M. immunogenum, M. intracellulare, M. interjectum, M. intermedium, M. kansasii, M. komossense, M. kubicae, M. lentiflavum, M. leprae, M. lepraemurium, M. luteum, M. madagascariense, M. mageritense, M. malmoense, M. marinum, M. microti, M. moriokaense, M. mucogenicum, M. murale, M. neoaurum, M. nonchromogenicum, M. novocastrense, M obuense, M. parafortuitum, M paratuberculosis, M. peregrinum, M. phage, M. phlei, M. porcinum, M. poriferae, M. pulveris, M. rhodesiae, M. scrofulaceum, M. senegalense, M. septicum, M. shimoidei, M. simiae, M. smegmatis, M. sphagni, M. szulgai, M. terrae, M. thermoresistibile, M. tokaiense, M. triplex, M. triviale, M. tuberculosis, M. tusciae, M. ulcerans, M. vaccae, M. wolinskyi, M. xenopi, Neisseria animalis, N. canis, N. cinerea, N. denitrificans, N. dentiae, N. elongata, N. flava, N. flavescens, N. gonorrhoeae, N. iguanae, N. lactamica, N. macacae, N. meningitidis ,N. mucosa, N. ovis, N. perflava, N. pharyngis var. flava, N. polysaccharea, N. sicca, N. subflava, N. weaveri, Pseudomonas aeruginosa, P. alcaligenes, P. chlororaphis, P. fluorescens, P. luteola, P. mendocina, P. monteilii, P. oryzihabitans, P. pertocinogena, P. pseudalcaligenes, P. putida, P. stutzeri, Salmonella bacteriophage, S. bongori, S. choleraesuis, S. enterica, S. enteritidis, S. paratyphi, S. typhi, S. typhimurium, S. typhimurium, S. typhimurium, S. typhimurium bacteriophage, Shigella boydii, S. dysenteriae, S. flexneri, S. sonnei, Staphylococcus arlettae, S. aureus, S. auricularis, S. bacteriophage, S. capitis, S. caprae, S. carnosus, S. caseolyticus, S. chromogenes, S. cohnii, S. delphini, S. epidermidis, S. equorum, S. felis, S. fleurettii, S. gallinarum, S. haemolyticus, S. hominis, S. hyicus, S. intermedius, S. kloosii, S. lentus, S. lugdunensis, S. lutrae, S. muscae, S. mutans, S. pasteuri, S. phage, S. piscifermentans, S. pulvereri, S. saccharolyticus, S. saprophyticus, S. schleiferi, S. sciuri, S. simulans, S. succinus, S. vitulinus, S. warneri, S. xylosus, Ureaplasma urealyticum, Yersinia aldovae, Y bercovieri, Y. enterocolitica, Y. frederiksenii, Y. intermedia, Y. kristensenii, Y. mollaretii, Y. pestis, Y. philomiragia, Y. pseudotuberculosis, Y rohdei, and Y. ruckeri.

The methods and compositions of the invention can also be used to treat or prevent bacterial infections by obligate intracellular bacteria, such as Anaplasma bovis, A. caudatum, A. centrale, A. marginale A. ovis, A. phagocytophila, A. platys, Bartonella bacilliformis, B. clarridgeiae, B. elizabethae, B. henselae, B. henselae phage, B. quintana, B. taylorii, B. vinsonii, Borrelia afzelii, B. andersonii, B. anserina, B. bissettii, B. burgdorferi, B. crocidurae, B. garinii, B. hermsii, B. japonica, B. miyamotoi, B. parkeri, B. recurrentis, B. turdi, B. turicatae, B. valaisiana, Brucella abortus, B. melitensis, Chlamydia pneumoniae, C. psittaci, C. trachomatis, Cowdria ruminantium, Coxiella burnetii, Ehrlichia canis, E. chaffeensis, E. equi, E. ewingii, E. muris, E. phagocytophila, E. platys, E. risticii, E. ruminantium, E. sennetsu, Haemobartonella canis, H. felis, H. muris, Mycoplasma arthriditis, M. buccale, M. faucium, M. fermentans, M. genitalium, M. hominis, M. laidlawii, M. lipophilum, M. orale, M. penetrans, M. pirum, M. neumoniae, M. salivarium, M. spermatophilum, Rickettsia australis, R. conorii, R. felis, R. helvetica, R. japonica, R. massiliae, R. montanensis, R. peacockii, R. rowazekii, R. rhipicephali, R. rickettsii, R. sibirica, and R. typhi.

Accordingly, the invention features methods of treating infections caused by the obligate and facultative intracellular bacteria above, among others.

The methods and compositions of the invention can also be used to treat or prevent fungal infections by a facultative intracellular fungus, such as Candida aaseri, C.acidothermophilum, C. acutus, C. albicans, C. anatomiae, C. apis, C. apis var. galacta, C. atlantica, C. atmospherica, C. auringiensis, C. bertae, C. berthtae var. chiloensis, C. berthetii, C. blankii, C. boidinii, C. boleticola, C. bombi, C. bombicola, C. buinensis, C. butyri, C. cacaoi, C. cantarellii, C. cariosilignicola, C. castellii, C. castrensis, C. catenulata, C. chilensis, C. chiropterorum, C. coipomensis, C. dendronema, C. deserticola, C. diddensiae, C. diversa, C. entomaea, C. entomophila, C. ergatensis, C. ernobii, C. ethanolica, C. ethanothermophilum, C. famata, C. fluviotilis, C. fragariorum, C. fragicola, C. friedrichii, C. fructus, C. geochares, C. glabrata, C. glaebosa, C. gropengiesseri, C. guilliermondii, C. guilliermondii var. galactosa, C. guilliermondii var. soya, C. haemulonii, C. halophila/C. versatilis, C. holmii, C. humilis, C. hydrocarbofumarica, C. inconspicua, C. insectalens, C. insectamans, C. intermedia, C. javanica, C. kefyr, C. krissii, C. krusei, C. krusoides, C. lambica, C. lusitaniae, C. magnoliae, C. maltosa, C. mamillae, C. maris, C. maritima, C. melibiosica, C. melinii, C.methylica, C. milleri, C. mogii, C. molischiana, C. montana, C. multis-gemmis, C. musae, C. naeodendra, C. nemodendra, C. nitratophila, C. norvegensis, C. norvegica, C. oleophila, C. oregonensis, C. osornensis, C. paludigena, C. parapsilosis, C. pararugosa, C. periphelosum, C. petrohuensis, C. petrophilum, C. philyla, C. pignaliae, C. pintolopesii var. pintolopesii, C. pintolopesii var. slooffiae, C. pinus, C. polymorpha, C. populi, C. pseudointermedia, C. quercitrasa, C. railenensis, C. rhagii, C. rugopelliculosa, C. rugosa, C. sake, C. salmanticensis, C. savonica, C. sequanensis, C. shehatae, C. silvae, C.silvicultrix, C. solani, C. sonorensis, C. sorbophila, C. spandovensis, C. sphaerica, C. stellata, C. succiphila, C. tenuis, C. terebra, C. tropicalis, C. utilis, C. valida, C. vanderwaltii, C. vartiovaarai, C. veronae, C. vini, C. wickerhamii, C. xestobii, C. zeylanoides, and Histoplasma capsulatum. Accordingly, the invention features a method of treating an infection by any of the facultative intracellular fungi above, among others.

Further, obligate intracellular protozoans can also be treated by intravenous administration of rifalazil as described herein. Obligate intracellular protozoans that may be treated by the methods of the invention include, for example, Brachiola vesicularum, B. connori, Encephalitozoon cuniculi, E. hellem, E. intestinalis, Enterocytozoon bieneusi, Leishmania aethiopica, L. amazonensis, L. braziliensis, L. chagasi, L. donovani, L. donovani chagasi, L. donovani donovani, L. donovani infantum, L. enriettii, L. guyanensis, L. infantum, L. major, L. mexicana, L. panamensis, L. peruviana, L. pifanoi, L. tarentolae, L. tropica, Microsporidium ceylonensis, M. africanum, Nosema connori, N. ocularum, N. algerae, Plasmodium berghei, P. brasilianum, P. chabaudi, P. chabaudi adami, P. chabaudi chabaudi, P. cynomolgi, P. falciparum, P. fragile, P. gallinaceum, P. knowlesi, P. lophurae, P. malariae, P. ovale, P. reichenowi, P. simiovale, P. simium, P. vinckei petteri, P. vinckei vinckei, P. vivax, P. yoelii, P. yoelii nigeriensis, P. yoelii yoelii, Pleistophora anguillarum, P. hippoglossoideos, P. mirandellae, P. ovariae, P. typicalis, Septata intestinalis, Toxoplasma gondii, Trachipleistophora hominis, T. anthropophthera, Vittaforma corneas, Trypanosoma avium, T. brucei, T. brucei brucei, T. brucei gambiense, T. brucei rhodesiense, T. cobitis, T. congolense, T. cruzi, T. Cyclops, T. equiperdum, T evansi, T. dionisii, T. godfreyi, T. grayi, T. lewisi, T. mega, T. microti, T. pestanai, T. rangeli, T. rotatorium, T. simiae, T. theileri, T. varani, T. vespertilionis, and T. vivax.

The rifalazil formulations described herein can further be used to treat or prevent viral infections.

Diseases associated with bacterial infections include, but are not limited to, multiple sclerosis (MS), rheumatoid arthritis (RA), inflammatory bowel disease (IBD), interstitial cystitis (IC), fibromyalgia (FM), autonomic nervous dysfunction (AND, neural-mediated hypotension); pyoderma gangrenosum (PG), chronic fatigue (CF) and chronic fatigue syndrome (CFS).

Several lines of evidence have led to the establishment of a link between bacterial infections and a broad set of inflammatory, autoimmune, and immune deficiency diseases. Thus, the present invention describes methods for treating chronic diseases associated with a persistent infection, such as autoimmune diseases, inflammatory diseases and diseases that occur in immuno-compromised individuals by treating the non-multiplying form of the infection in an individual in need thereof, by administering a rifamycin antibiotic described herein, or such a rifamycin in conjunction with an antibiotic effective against multiplying bacteria. Progress of the treatment can be evaluated, using the diagnostic tests known in the art, to determine the presence or absence of the bacteria. Physical improvement in the conditions and symptoms typically associated with the disease to be treated can also be evaluated. Based upon these evaluating factors, the physician can maintain or modify the anti-bacterial therapy accordingly.

The therapies described herein can be used for the treatment of chronic immune and autoimmune diseases when patients are demonstrated to have a bacterial infection. These diseases include, but are not limited to, chronic hepatitis, systemic lupus erythematosus, arthritis, thyroidosis, scleroderma, diabetes mellitus, Graves' disease, Beschet's disease, and graft versus host disease (graft rejection). The therapies of this invention can also be used to treat any disorders in which a bacterial infection is a factor or co-factor.

Thus, the present invention can be used to treat a range of disorders in addition to the above immune and autoimmune diseases when demonstrated to be associated with chlamydial infection by the methods of detection described herein; for example, various infections, many of which produce inflammation as primary or secondary symptoms, including, but not limited to, sepsis syndrome, cachexia, circulatory collapse and shock resulting from acute or chronic bacterial infection, acute and chronic parasitic and/or infectious diseases from bacterial, viral or fungal sources, such as a HIV, AIDS (including symptoms of cachexia, autoimmune disorders, AIDS dementia complex and infections) can be treated.

Among the various inflammatory diseases, there are certain features that are generally agreed to be characteristic of the inflammatory process. These include fenestration of the microvasculature, leakage of the elements of blood into the interstitial spaces, and migration of leukocytes into the inflamed tissue. On a macroscopic level, this is usually accompanied by the familiar clinical signs of erythema, edema, tenderness (hyperalgesia), and pain. Inflammatory diseases, such as chronic inflammatory pathologies and vascular inflammatory pathologies, including chronic inflammatory pathologies such as aneurysms, hemorrhoids, sarcoidosis, chronic inflammatory bowel disease, ulcerative colitis, and Crohn's disease and vascular inflammatory pathologies, such as, but not limited to, disseminated intravascular coagulation, atherosclerosis, and Kawasaki's pathology are also suitable for treatment by methods described herein. The invention can also be used to treat inflammatory diseases such as coronary artery disease, hypertension, stroke, asthma, chronic hepatitis, multiple sclerosis, peripheral neuropathy, chronic or recurrent sore throat, laryngitis, tracheobronchitis, chronic vascular headaches (including migraines, cluster headaches and tension headaches) and pneumonia when demonstrated to be pathogenically related to a bacterial infection.

Treatable disorders when associated with a bacterial infection also include, but are not limited to, neurodegenerative diseases, including, but not limited to, demyelinating diseases, such as multiple sclerosis and acute transverse myelitis; extrapyramidal and cerebellar disorders, such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; progressive supranucleo palsy; cerebellar and spinocerebellar disorders, such as astructural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and Machado-Joseph)); and systemic disorders (Refsum's disease, abetalipoprotemia, ataxia, telangiectasia, and mitochondrial multi-system disorder); demyelinating core disorders, such as multiple sclerosis, acute transverse myelitis; disorders of the motor unit, such as eurogenic muscular atrophies (anterior horn cell degeneration, such as myotrophic lateral sclerosis, infantile spinal muscular atrophy and juvenile spinal uscular atrophy); Alzheimer's disease; Down's Syndrome in middle age; Diffuse ewy body disease; senile dementia of Lewy body type; Wernicke-Korsakoff syndrome; chronic alcoholism; Creutzfeldt-Jakob disease; subacute sclerosing anencephalitis, Hallerrorden-Spatz disease; and dementia pugilistica.

It is also recognized that malignant pathologies involving tumors or other alignancies, such as, but not limited to leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or mycosis fungoides)); carcinomas (such as colon carcinoma) and metastases thereof; cancer-related angiogenesis; infantile hemangiomas; and alcohol-induced hepatitis. Ocular neovascularization, psoriasis, duodenal ulcers, angiogenesis of the female reproductive tract, can also be treated when demonstrated by the diagnostic procedures described herein to be associated with a bacterial infection.

Packaging

The compositions of the invention may be packaged together with instructions for the intravenous administration of a rifalazil. Typically, the instructions will also include the dosage and rate of administration. In some instances, instructions may be included on a label or on a package insert accompanying an intravenous pharmaceutical formulation containing rifalazil.

The method of the invention can be incorporated into a prepackaged therapeutic regimen designed to deliver a specific dose of rifalazil over a specific period of time to a human patient. For example, a sufficient amount of rifalazil can be administed as a “push” over ten to sixty minutes to produce a desired blood level and the remainder of the dose would be administered over a period of up to a total of 24 hours at such a rate that the blood level would remain constant. In this manner rifalazil may be intravenously administered every day, every other day, every third day for a period of up twelve days, or weekly for a period of three to sixteen weeks with continuing doses available orally using a weekly regimen if desired.

The compositions can also be packaged as a concentrate including rifalazil and micelle-forming excipient. The concentrate optionally includes some water. For example, the concentrate can be less than 40%, 20%, 10%, 5%, or even 1% water by volume. The concentrate contains greater than 100 μg/mL, 1 mg/mL, 5 mg/mL, 10 mg/mL, or 20 mg/mL of rifalazil. Concentrates are formulated for intravenous adminstration by the addition of water, which may include other pharmaceutically acceptable excipients, such as buffer or saline, in an amount necessary to achieve the concentration of rifalazil to be administered.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLE 1

### SOLUBILITY STUDIES

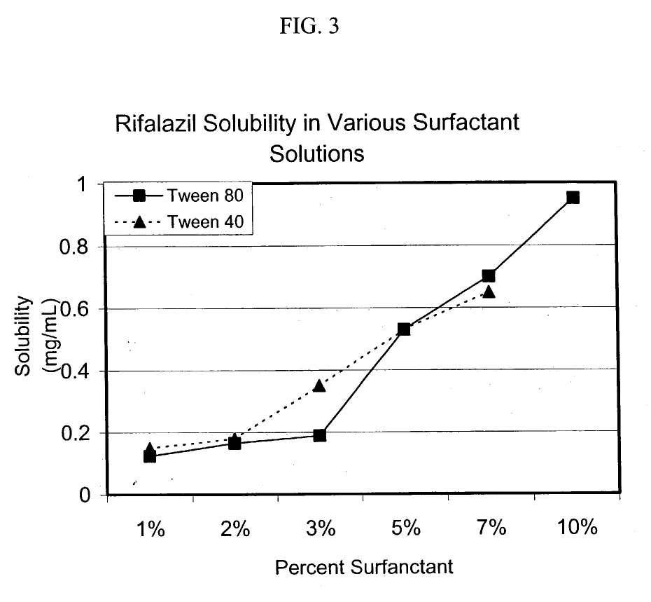

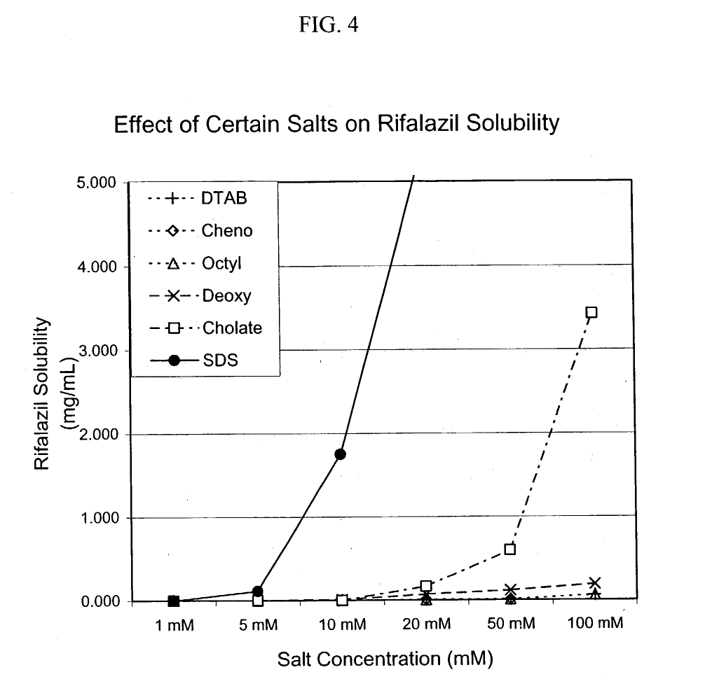

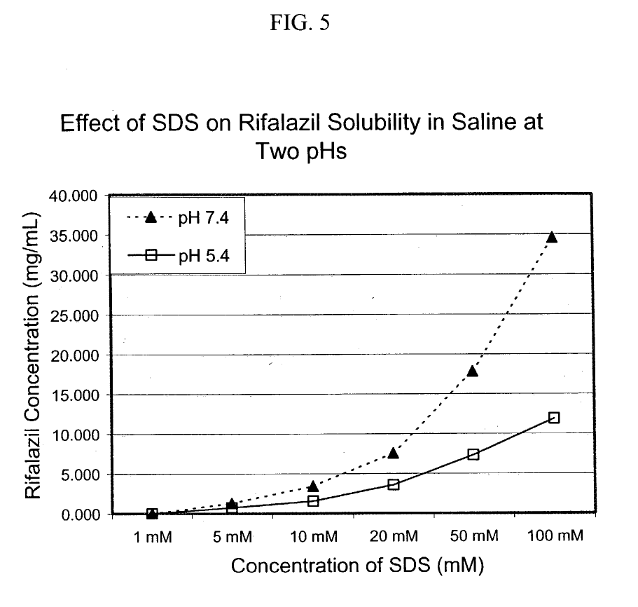

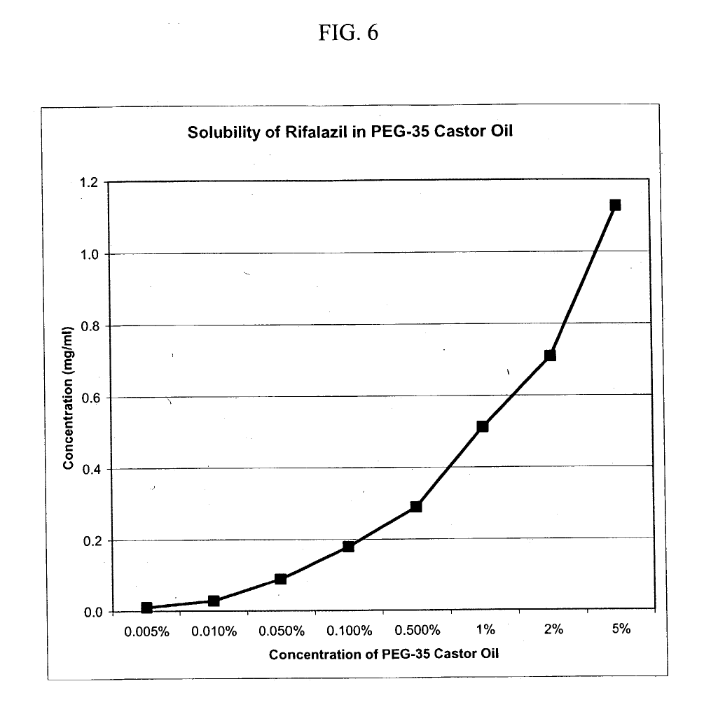

In order to determine its solubility, to a vial containing solid rifalazil was added a volume of aqueous test solution. The vial was capped and sonicated for about an hour and the remaining undissolved solids were left to settle overnight. The vial was then vortexed for 1 minute, sonicated for another 30 minutes, and centrifuged for 1 hour at 3500 RPM. The supernatent was separated from undissolved rifalazil. The concentration of rifalazil in the test solution was determined by UV-Vis absorbance using a suitable spectrophotometer. Using this method, the solubility of rifalazil was determined as a function of pH (see FIG. 1); in PEG 400-water, propylene glycol-water, and ethanol-water mixtures (see FIG. 2); in Tween-40-water and Tween-80-water mixtures (see FIG. 3); in dodecyltrimethylammonium bromide-water, sodium chenodeoxycholate-water, sodium Octylsulfate-water, sodium deoxycholate-water, sodium cholate-water, and sodium dodecylsulfate-water mixtures (see FIG. 4); in sodium dodecylsulfate- water mixtures at pH 5.4 and 7.4 (see FIG. 5); and in PEG-35 castor oil-water mixtures (see FIG. 6).

### EXAMPLE 2

### Formulation of Rifalazil for Intravenous Administration

To 250 mL water containing 10 mM phosphate buffer (pH=7.6), 1.0% (v/v) PEG-35 castor oil, and 0.9% (w/v) NaCl was added from 2.5 mg to 25 mg of rifalazil. The solution was gently agitated until no undissolved solids remained.

### EXAMPLE 3

### Stability Studies

Rifalazil solutions containing the micelle-forming excipient PEG-35 castor oil, prepared as described in Example 2, and without a micelle-forming excipient (15% ethanol, phosphate buffer pH=7.6, 0.9% NaCl) were monitored for stability against the hydrolytic degradation of rifalazil at temperatures of 25° C., 40° C., 50° C., and 60° C. (see FIGS. 7 and 8). The degradation was monitored for several days by HPLC for the disappearance of rifalazil or the appearance of des-acetyl rifalazil, the degradation product that results from the hydrolysis of rifalazil. The presence of a micelle-forming excipient inhibits the hydrolytic degradation of rifalazil, as shown in FIGS. 7 and 8.

### EXAMPLE 4

### In Vitro Data

The MIC (minimum inhibitory concentration) of rifalazil against S. aureus was determined by the broth microdilution method. A vehicle prepared as described in Example 2 was diluted in Cation Adjusted Mueller-Hinton (CAMH) broth to create working solutions that were 4× the maximum concentration to be tested on each microtiter plate. The 4× working solutions were added to the wells of the 96 well microtiter plate and serially diluted two-fold across 11 wells into CAMH.

To prepare the test strain inoculum, 3-5 colonies of S. aureus from a pure 18-24 hour culture grown on a primary agar plate were emulsified into demineralized water and adjusted to a 0.5 McFarland turbidity standard using a calibrated nephelometer. This suspension was then diluted further (1:100) into CAMH broth to yield an inoculum suspension containing approximately 10⁶ CFU/ml. Aliquots of the inoculum suspension were added to the rifalazil-containing wells to yield a final concentration in the well of 1-8×10⁵ CFU/ml. The microtiter plates were incubated at 35-37° C. for 20-24 hours. The MIC was read as the lowest concentration of the compound that inhibits visible growth.

### Example 5

### In Vivo Data

Rifalazil was evaluated in a murine model of bacterial infection in which female mice that weighed approximately 20 g were challenged by intraperitoneal injection of bacterial cells of S. aureus Smith strain from a log phase broth culture, sufficient in number to kill non-treated control mice within 24 to 48 hours. 20 Rifalazil was tested using the procedure described by Weiss et al., Antimicrobial Agents and Chemotherapy 43:460-464 (1999).

Rifalazil was administered to mice 30 minutes after inoculation with bacteria, either by intravenous route, using the vehicle prepared as described in Example 2, or by oral gravage, using the same vehicle. Mice were found to have 25 survived if they lived for three days following treatment. The MIC, intravenous ED₅₀, and oral ED₅₀ are provided in Table 1.

TABLE 1
Effective Dose of Rifalazil (μg/mL)

| MIC | IV ED₅₀ | Oral ED₅₀ |
|---|---|---|
| 0.015 | 0.053 | 0.098 |

### Other Embodiments

All publications, patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

What we claim is:

1. An aqueous solution of rifalazil suitable for intravenous administration to a human, wherein said solution has a rifalazil concentration of between 10 to 10,000 μg/mL.

2. The solution of claim 1, wherein said rifalazil concentration is between 50 and 10,000 μg/mL.

3. The solution of claim 2, wherein said rifalazil concentration is between 100 and 2,000 82 g/mL.

4. The solution of claim 1, further comprising an excipient selected from the group consisting of polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, and ionic surfactants.

5. The solution of claim 4, wherein said excipient is selected from the group consisting of sodium lauryl sulfate, polyoxyl-40 stearate, PEG-3 castor oil, PEG-5 castor oil, PEG-9 castor oil, PEG-16 castor oil, PEG-20 castor oil, PEG-23 castor oil, PEG-30 castor oil, PEG-35 castor oil, PEG-38 castor oil, PEG-40 castor oil, PEG-50 castor oil, PEG-60 castor oil, PEG-100 castor oil, PEG-200 castor oil, PEG-5 hydrogenated castor oil, PEG-7 hydrogenated castor oil, PEG-10 hydrogenated castor oil, PEG-20 hydrogenated castor oil, PEG-25 hydrogenated castor oil, PEG-30 hydrogenated castor oil, PEG-40 hydrogenated castor oil, PEG-45 hydrogenated castor oil, PEG-50 hydrogenated castor oil, PEG-60 hydrogenated castor oil, PEG-80 hydrogenated castor oil, and PEG-100 hydrogenated castor oil.

6. The solution of claim 5, wherein said excipient is PEG-35 castor oil.

7. An aqueous composition for inhibiting the hydrolytic decomposition of rifalazil dissolved therein, said composition comprising rifalazil, water, and a micelle-forming excipient.

8. A method for inhibiting the hydrolytic decomposition of rifalazil, said method comprising formulating said rifalazil in an aqueous solution containing a micelle-forming excipient.

9. A method of treating a disease in a human, said method comprising administering rifalazil intravenously to said human in an amount effective to treat said disease.

10. The method of claim 9, wherein said administration of rifalazil comprises an intravenous infusion of between 1 and 48 mg of rifalazil to said human over a period of 4 to 24 hours.

11. The method of claim 10, wherein said administration of rifalazil comprises:
(a) a bolus injection of between 2 and 25 mg of rifalazil over 10 to 60 minutes, and
(b) following step (a), a slow infusion of between 0.1 and 2 mg per hour for up to 24 hours.

12. The method of claims 10 or 11, wherein said intravenous administration is repeated.

13. The method of claim 9, wherein said rifalazil is administered in an amount necessary to maintain a rifalazil concentration of between 2 and 100 ng/mL in the plasma of said human for a period greater than 5 hours.

14. The method of claim 13, wherein said rifalazil is administered in an amount necessary to maintain a rifalazil concentration of between 2 and 40 ng/mL in the plasma of said human for a period greater than 24 hours.

15. The method of any of claims 9, 10, or 13, further comprising the administration of a second antibiotic.

16. The method of claim 9, wherein said disease is selected from the group consisting of a community-acquired pneumonia, upper and lower respiratory tract infection, skin and soft tissue infection, bone and joint infection, hospital-acquired lung infection, acute bacterial otitis media, bacterial pneumonia, complicated infection, noncomplicated infection, pyelonephritis, intra-abdominal infection, deep-seated abcess, bacterial sepsis, central nervous system infection, bacteremia, wound infection, peritonitis, meningitis, infections after burn, urogenital tract infection, gastro-intestinal tract infection, pelvic inflammatory disease, endocarditis, and intravascular infection.

17. The method of claims 9, wherein said disease is selected from the group consisting of atherosclerosis, multiple sclerosis, rheumatoid arthritis, diabetes, Alzheimer's disease, asthma, cirrhosis of the liver, psoriasis, meningitis, cystic fibrosis, cancer, and osteoporosis.

18. The method of claim 9, wherein said rifalazil is administered for prophylaxis against an infection resulting from a surgical procedure or implantation of a prosthetic device.

19. The method of claim 9, wherein said disease is a protozoan, bacterial, viral, or fungal infection.

20. The method of claim 19, wherein said infection is by Gram-positive bacterium.

21. The method of claim 20, wherein said bacterium is a Gram-positive coccus.

22. The method of claim 21, wherein said Gram-positive coccus is drug-resistant.

23. The method of claim 19, wherein said infection is by a bacterium selected from the group consisting of S. aureus, S. epidermidis, S. pneumoniae, S. pyogenes, Enterococcus spp., M. catarrhalis, and H. influenzae.

24. The method of claim 19, wherein said infection is an intracellular infection.

25. The method of claim 24, wherein said intracellular infection is caused by an obligate intracellular bacterium.

26. The method of claim 25, wherein said obligate intracellular bacterium is selected from the group consisting of Anaplasma bovis, A. caudatum, A. centrale, A. marginale A. ovis, A. phagocytophila, A. platys, Bartonella bacilliformis, B. clarridgeiae, B. elizabethae, B. henselae, B. henselae phage, B. quintana, B. taylorii, B. vinsonii, Borrelia afzelii, B. andersonii, B. anserina, B. bissettii, B. burgdorferi, B. crocidurae, B. garinii, B. hermsii, B. japonica, B. miyamotoi, B. parkeri, B. recurrentis, B. turdi, B. turicatae, B. valaisiana, Brucella abortus, B. melitensis, Chlamydia pneumoniae, C. psittaci, C. trachomatis, Cowdria ruminantium, Coxiella burnetii, Ehrlichia canis, E. chaffeensis, E. equi, E. ewingii, E. muris, E. phagocytophila, E. platys, E. risticii, E. ruminantium, E. sennetsu, Haemobartonella can is, H. felis, H. muris, Mycoplasma arthriditis, M. buccale, M. faucium, M. fermentans, M. genitalium, M. hominis, M. laidlawii, M. lipophilum, M. orale, M. penetrans, M. pirum, M. pneumoniae, M. salivarium, M. spermatophilum, Rickettsia australis, R. conorii, R. felis, R. helvetica, R. japonica, R. massiliae, R. montanensis, R. peacockii, R. prowazekii, R. rhipicephali, R. rickettsii, R. sibirica, and R. typhi.

27. The method of claim 24, wherein said intracellular infection is caused by an obligate intracellular protozoan.

28. The method of claim 27, wherein said obligate intracellular protozoan is selected from the group consisting of Brachiola vesicularum, B. connori, Encephalitozoon cuniculi, E. hellem, E. intestinalis, Enterocytozoon bieneusi, Leishmania aethiopica, L. amazonensis, L. braziliensis, L. chagasi, L. donovani, L. donovani chagasi, L. donovani donovani, L. donovani infantum, L. enriettii, L. guyanensis, L. infantum, L. major, L. mexicana, L. panamensis, L. peruviana, L. pifanoi, L. tarentolae, L. tropica, Microsporidium ceylonensis, M. africanum, Nosema connori, Nosema ocularum, N. algerae, Plasmodium berghei, P. brasilianum, P. chabaudi, P. chabaudi adami, P. chabaudi chabaudi, P. cynomolgi, P. falciparum, P. fragile, P. gallinaceum, P. knowlesi, P. lophurae, P. malariae, P. ova le, P. reichenowi, P. simiovale, P. simium, P. vinckei petteri, P. vinckei vinckei, P. vivax, P. yoelii, P. yoelii nigeriensis, P. yoelii yoelii, Pleistophora anguillarum, P. hippoglossoideos, P. mirandellae, P. ovariae, P. typicalis, Septata intestinalis, Toxoplasma gondii, Trachipleistophora hominis, T. anthropophthera, Vittaforma corneae, Trypanosoma avium, T. brucei, T. brucei brucei, T. brucei gambiense, T. brucei rhodesiense, T. cobitis, T. congolense, T. cruzi, T. cyclops, T. equiperdum, T. evansi, T. dionisii, T. godfreyi, T. grayi, T. lewisi, T. mega, T. microti, T. pestanai, T. rangeli, T. rotatorium, T. simiae, T theileri, T. varani, T. vespertilionis, and T. vivax.

29. The method of claim 24, wherein said intracellular infection is caused by an intracellular fungus.

30. The method of claim 29, wherein said intracellular fungus is Histoplasma capsulatum or a species of the genus Candida.

31. The method of claim 24, wherein said intracellular infection is caused by a virus.

32. The method of claim 19, said method further comprising co-administering an effective therapeutic amount of an antifungal agent, antiviral agent, antibacterial agent, or antiprotozoan agent.

33. A method of treating an infection by multi-drug resistant bacteria in a human, said method comprising the intravenous administration of rifalazil to said human in an amount effective to treat said infection.

34. The method of claim 33, wherein said multi-drug resistant bacteria are penicillin-resistant, methicillin-resistant, quinolone-resistant, macrolide-resistant, or vancomycin-resistant bacteria.

35. The method of claim 34, wherein said bacteria are selected from the group consisting of Streptococcus pneumoniae, Staphylococcus aureus, Streptococcus pyogenes, and Enterococcus spp.

36. A method for treating or preventing the development of an atherosclerosis-associated disease in a human patient in need thereof, said method comprising the intravenous administration of rifalazil to said patient in an amount effective to treat or prevent the development of said atherosclerosis-associated disease in said patient.

37. The method of claim 36, further comprising the step of administering to said patient an anti-inflammatory agent, antibacterial agent, platelet aggregation inhibitor, anticoagulant, antipyretic, or lipid-lowering agent.

38. The method of claim 37, wherein said patient is administered an anti- inflammatory agent.

39. The method of claim 38, wherein said anti-inflammatory agent is ibuprofen, meloxicam, celecoxib, rofecoxib, aspirin, dexamethasone, methylprednisolone, prednisolone, or prednisone.

40. The method of claim 37, wherein said patient is administered an antibacterial agent.

41. The method of claim 40, wherein said antibacterial agent is azithromycin, clarithromycin, erythromycin, gatifloxacin, levofloxacin, amoxicillin, or metronidazole.

42. The method of claim 37, wherein said lipid-lowering agent is a statin.

43. The method of claim 42, wherein said statin is atorvastatin, rosuvastatin, lovastatin, simvastatin, pravastatin, cerivastatin, or fluvastatin.

44. The method of claim 36, wherein said atherosclerosis-associated disease is coronary artery disease, myocardial infarction, angina pectoris, stroke, cerebral ischemia, intermittent claudication, gangrene, mesenteric ischemia, temporal arteritis, or renal artery stenosis.

45. The method of claim 36, wherein, prior to administration of said compound, said patient is diagnosed as having said atherosclerosis-associated disease.

46. A method of reducing the level of C-reactive protein in a human patient identified as having increased levels of C-reactive protein, said method comprising the intravenous administration of rifalazil to said patient in an amount effective to reduce the level of C-reactive protein.

47. The method of claim 46, wherein said method further comprises the step of periodically monitoring the level of C-reactive protein in said patient following administration of said compound.

48. A method for reducing Chlamydia pneumoniae replication in macrophages or foam cells in a human patient in need thereof, said method comprising the intravenous administration of rifalazil to said patient in an amount effective to reduce Chlamydia pneumoniae replication in macrophages or foam cells in said patient.

49. A method for treating a persistent Chlamydia pneumoniae infection in macrophages or foam cells in a human patient, said method comprising the intravenous administration of rifalazil to said patient in an amount effective to treat said Chlamydia pneumoniae infection in macrophages or foam cells in said patient.

50. A method for treating an infection of a bacterium having a multiplying form and a non-multiplying form, said method comprising administering to a patient (i) rifalazil; and (ii) a second antibiotic effective against the multiplying form of said bacterium, wherein said rifalazil is administered intravenously in an amount and for a duration effective to treat the non-multiplying form of said bacterium and the second antibiotic is administered in an amount and for a duration effective to treat said multiplying form of said bacterium.

51. The method of claim 50, wherein said antibiotic effective against said multiplying form of said bacterium is administered to said patient in an amount and for a duration to reduce the presence of said bacterium in said patient to less than about 10⁶ organisms/mL; and said rifalazil is then administered intravenously to said patient in an amount and for a duration effective to reduce the presence of said bacterium to or below a level indicative that said infection has been treated.

52. A method of eradicating non-multiplying bacteria not eradicated in a patient following treatment with a first antibiotic, said method comprising administering rifalazil intravenously to said patient in an amount and for a duration effective to eradicate said non-multiplying bacteria in said patient.

53. A method of treating a patient diagnosed as having a chronic disease associated with a bacterial infection caused by bacteria capable of establishing a non-multiplying form phase, said method comprising the step of administering rifalazil intravenously to said patient, wherein said administering is for a duration and in an amount effective to treat said patient.

54. The method of claim 53, wherein said chronic disease is an inflammatory disease.

55. The method of claim 54, wherein said inflammatory disease is selected from the group consisting of asthma, coronary artery disease, arthritis, conjunctivitis, lymphogranuloma venerum, cervicitis, and salpingitis.

56. The method of claim 53, wherein said chronic disease is an autoimmune disease.

57. The method of claim 56, wherein said autoimmune disease is selected from the group consisting of systemic lupus erythematosus, diabetes mellitus, and graft versus host disease.

58. The method of claim 53, wherein said chronic disease is atherosclerosis.

59. A method of treating the cryptic phase of a bacterial infection, said method comprising the step of administering rifalazil intravenously to a patient, wherein said administering is for a duration and in an amount effective to treat said cryptic phase of said bacterial infection.

60. A pharmaceutical formulation comprising rifalazil for intravenous administration, wherein said formulation is packaged with a label or package insert providing instructions for the use of said formulation, wherein the instructions describe an intravenous dosing regimen.

61. A concentrate comprising rifalazil and a micelle-forming excipient, wherein said concentrate comprises less than 40% water by volume and greater than 100 μg/mL of rifalazil.

62. The concentrate of claim 61, wherein said concentrate comprises less than 5% water by volume and greater than 1 mg/mL of rifalazil.
